SEQUENCE LISTING (25 sequences)

SEQ ID NO: 1 - DNA, 927 bp, Moraxella catarrhalis
atgaaatcaatcaaaacttttaccgcctgtcttacgatgtgcttagccttggttgggtgcggtcagcagaccaaagaagacatcaacgcacaggatactcattctccaaaaaaactgtctgttgtcacaacttttactgtcattgctgacattgcccagaatgttgctggagaggctgccgatgttcaatccattaccaaggcaggtgctgaaattcatgaatatgaacccacgcctcaagatgtggttaaagcccaaaaagcagatttgattttatggaatggacttaatctagagctttggtttgaaaaattttatcatgataccagcaatgtgcctgccgttgttgtgacacagggtatcacaccgattaacatcactgaaggtgcttataaagacatgccaaacccacatgcttggatgtcgccaagtaatgccttgatttatgttgagaatatcaaaaatgcactcattaagcaagacccagccaatcaggaagtttataccaaaaatgctgagcagtacagtgcaaaaattaaagctatggacgctccactgcgtgccaaattatcacaaatccctgaaaatcagcgttggttggtaaccagtgaaggtgcgtttagctacttggccaacgattacggtcttaaagaagcttatttgtggccaattaatgccgaacagcaaggctcgcctcaacaagtcaaatcattgattgatacggtacgcagcaataacattcctgtggtatttagtgaaagtacaatttcagacaaaccagcaaaacaagttgccaaagaaacaggtgccaaatatggcggtgtactttatgttgattcactgtctgaagctggcggtcctgtaccaacttatcttgacttattacaaaccaccgtctcaaccatcgccagcggttttgaaaagtga

SEQ ID NO: 2 - protein, 308 aa, Moraxella catarrhalis
MKSIKTFTACLTMCLALVGCGQQTKEDINAQDTHSPKKLSVVTTFTVIADIAQNVAGEAADVQSITKAGAEIHEYEPTPQDVVKAQKADLILWNGLNLELWFEKFYHDTSNVPAVVVTQGITPINITEGAYKDMPNPHAWMSPSNALIYVENIKNALIKQDPANQEVYTKNAEQYSAKIKAMDAPLRAKLSQIPENQRWLVTSEGAFSYLANDYGLKEAYLWPINAEQQGSPQQVKSLIDTVRSNNIPVVFSESTISDKPAKQVAKETGAKYGGVLYVDSLSEAGGPVPTYLDLLQTTVSTIASGFEK

SEQ ID NO: 3 - DNA, 1080 bp, Moraxella catarrhalis
atgataacattgcttaaaaaaacaacactgctggcaacagcatttggtatcttttttggcttaactgcttgctcaaacaataccaaaacttccatccaaaatactgacacgacagccagtagtacagaagctaatcaagatccaattgagcttttgaatgtttcttacgatgtgtctcgagatttttataaagcgtataatgaagtttttataaatcattatttgaccaactacccaaataccaaaattaagatcaagcagtctcatggtggctcaagtaagcaagcactttcggtaaaaaatggactgcaagcggatattgttaccatgaatcaaggctctgatattgagctattggctgatgccaaattggtggatggcaattggcgacacgcattcccaaaccatgccattcctttcaccagtactatcgtatttttggtacgagaaggcaaccccaaaggcattaaagattggaatgacttggcaacaagtggcatagatattatcatcgccaacccaaaaacctctggtaacggtcgctatgcgtttttgggtgcttatggctatggcttgaatgcttatcataatgacgaagctcaaactcatgcgttcgtcaaagcacttttaagccatgttaaagtatttgaaaatggtgggcgtgcggcaacaacaacctttgcccagcgtggcatcggtgatgtcttaatcacctttgaaaatgaagccaatgtgattaagagtaaatttggcaatgtggatattatttacccaagctacacagtgaatgcagaaaatccgattgctgtggtgaaatctgttaccgacaacaaaggcacaaccaaagctgcaaccgaatacctcaatcatctgtacagcgatgaagctcaagagcttgccacgaagtttttcttaagaccaagcagccccgatatcctagccaaacacgccgataaattccctgtcatgacaaccttttttcctaatgatgtctttggagactgggcaagtatcatgaaaacttattttagtgatggcggcttatacgataagcttgccgtccaagctcaaaatcagtaa

SEQ ID NO: 4 - protein, 359 aa, Moraxella catarrhalis
MITLLKKTTLLATAFGIFFGLTACSNNTKTSIQNTDTTASSTEANQDPIELLNVSYDVSRDFYKAYNEVFINHYLTNYPNTKIKIKQSHGGSSKQALSVKNGLQADIVTMNQGSDIELLADAKLVDGNWRHAFPNHAIPFTSTIVFLVREGNPKGIKDWNDLATSGIDIIIANPKTSGNGRYAFLGAYGYGLNAYHNDEAQTHAFVKALLSHVKVFENGGRAATTTFAQRGIGDVLITFENEANVIKSKFGNVDIIYPSYTVNAENPIAVVKSVTDNKGTTKAATEYLNHLYSDEAQELATKFFLRPSSPDILAKHADKFPVMTTFFPNDVFGDWASIMKTYFSDGGLYDKLAVQAQNQ

SEQ ID NO: 5 - DNA, 804 bp, Moraxella catarrhalis
gtgatcatgacaaccgtgttaaaatttaccacagccgcattttgtgtttctttacttgctgcttgtagcaatgagtctacgaccaataccaaaactactgctgcacctaacagctcgacctcacaaaaagaattgattatcgctactgaatctagctttaagccatttagctatcttgacaagcaaggtcaattggtcggctttgagattgatttggcaaatgcactatgcaagcagatgcaagcagattgtcaaatcgtaagccaagactgggacagtcttattccaagcttgaacgccaataagtctgatgccatcatggcgggtatgtccattaccgaagaacgcctaaatgcagtggactttagtgagccttattttgataatacgcttgttttgattggtaagaaagacaataccgccaccatcaatgacatcgctggtaaagctgttgcaacgcagcaggcaactgtttcagcggaatatcttcaaaaacattatccagacgccaacatcaaaacttatgataagcaagacaacgcttatcttgatttgaccgctgggcgtgttgaatttatgttgtctgatgtcgtgcctgctactgactggcttgctactgaagctggctctgaatttgagatcaaaggcatgcccattgatatcggtgataaagtcggtatcgctgtgcgtaaagatagcagcctaaaagcggattttgacgctgcacttgccagcctaaaaactaatggcaaatatgatgagattgttgccaaatactttaacagccaagcattaggtcaataa

SEQ ID NO: 6 - protein, 267 aa, Moraxella catarrhalis
VIMTTVLKFTTAAFCVSLLAACSNESTTNTKTTAAPNSSTSQKELIIATESSFKPFSYLDKQGQLVGFEIDLANALCKQMQADCQIVSQDWDSLIPSLNANKSDAIMAGMSITEERLNAVDFSEPYFDNTLVLIGKKDNTATINDIAGKAVATQQATVSAEYLQKHYPDANIKTYDKQDNAYLDLTAGRVEFMLSDVVPATDWLATEAGSEFEIKGMPIDIGDKVGIAVRKDSSLKADFDAALASLKTNGKYDEIVAKYFNSQALGQ

SEQ ID NO: 7 - DNA, 2039 bp, Moraxella catarrhalis
atgaaaaaaacgaagttatttgccacagtaggggcagcagtattatcagcatctattttagctgcatgcagcaataatagcacgacagcatcacaaggtggcggtgatttgaccacctataaatatgtgttttcaggtgacccaaaatctttagactatatcttagccaaccaagctgttactgccgatgttaccactcagatggttgatggtttgctagaaaatgatgaatatggcaaccttgtcccttctttggcgactgattggagcgtttcagaagatggattaacttatacctacaccttgcgtgatggggtattttggtacacttcagatggcgaagaatatgcgcccgttactgcccatgactttgtgacaggtctaaaacatgccgttgacggtaaatctgatgcactttatgtggttgaagattctgtaaaaaacctaaaagcctataaagaaggtaaagtgaattgggaagaggttggcgttaaggctttagatgataaaactgtacaatacacgctaaaccaaccagaatcatattggaattctaaagtgacctatagcgtcctattccctgtgaatgctaagtttttacaatctaaaggtaaagattttggtgctttagatccttcatctattttggtcaatggtgcttactttttaagtgcctatgcatcaaaatctttgatggaatttactaaaaatgataattattgggatgctgacaatgttcatgtacagtctgtcaagctaacctataccgatggctcagatcctggttcgtattatcgtaactttgataaaggcgagtttagcgttgcacgcttatatccaaatgacccaacttaccaagctgccagtgaaaaatatcaagataacatcgtttatggcttgatagatggtacaacttattatttcacctttaacctaaatcgttctgcttttgccaatagcacaaaaacgccagagcaacaagaatctgccaaaaaggcaatgctgaacaaagatttccgtcaagcggttatgtttgcacttgatcgtgcagcttatcaagcacaaactgtcggtgaggaagccaaaaccaaggccttgcgtaatatgctggtaccgccaacttttgtgtctgccgatggtgaagactttgggcaaatggtgaaaaaagacttggtaggctatggtgcagaatggcaagatgtcgatttgtcagatagccaagatggtctatacaatccacaaaaagccaaagaagagtttgctaaagccagacaaactttagaagctcaaggcgtgactttccctatctatttggattttccaattgaccaagcagattcgaacagagttcagcaagctcaatcattcaagcaatcggttgaggcctcattgggtcaagaaaatattattatcaatgtcattgaaaccgagacctcaacttatgagtctcaaggctactatgctgaatcgccagagcagcaagattatgatattatgatggcaggttggggcccggattatcaagatccacgcacctatttggatatcatgagtcctattgatggtgccatgctacaaaaaacaggtatccacagaggtggcgataaagctttagttaaacaagttggtttggatacttatcaaacactgcttaatcaggcatcggtcatctctaatgataacagtgcgcgttataatgcctatgccaaagcacaggcattactgcttgacagtgctttacaaatcccaatggttgctattggtggtgtaccaagagtttctaaaggcgtaccatttagcggatcattttcttgggcgggcaataaaggcggtagctggtataagagattaaaactacaagcacagcctatcacgactgagcaatatgagaaagcctatcaagcatggcaaagtgaaaaatcggcttctaatgccaaatatgctgacagccttgttaatcgtgtcaaaaaatcagatacagcagcaagcgatgctgcagcaactgatgctgcaaccacggatacgacaacagcgaattg

SEQ ID NO: 8 - protein, 679 aa, Moraxella catarrhalis
MKKTKLFATVGAAVLSASILAACSNNSTTASQGGGDLTTYKYVFSGDPKSLDYILANQAVTADVTTQMVDGLLENDEYGNLVPSLATDWSVSEDGLTYTYTLRDGVFWYTSDGEEYAPVTAHDFVTGLKHAVDGKSDALYVVEDSVKNLKAYKEGKVNWEEVGVKALDDKTVQYTLNQPESYWNSKVTYSVLFPVNAKFLQSKGKDFGALDPSSILVNGAYFLSAYASKSLMEFTKNDNYWDADNVHVQSVKLTYTDGSDPGSYYRNFDKGEFSVARLYPNDPTYQAASEKYQDNIVYGLIDGTTYYFTFNLNRSAFANSTKTPEQQESAKKAMLNKDFRQAVMFALDRAAYQAQTVGEEAKTKALRNMLVPPTFVSADGEDFGQMVKKDLVGYGAEWQDVDLSDSQDGLYNPQKAKEEFAKARQTLEAQGVTFPIYLDFPIDQADSNRVQQAQSFKQSVEASLGQENIIINVIETETSTYESQGYYAESPEQQDYDIMMAGWGPDYQDPRTYLDIMSPIDGAMLQKTGIHRGGDKALVKQVGLDTYQTLLNQASVISNDNSARYNAYAKAQALLLDSALQIPMVAIGGVPRVSKGVPFSGSFSWAGNKGGSWYKRLKLQAQPITTEQYEKAYQAWQSEKSASNAKYADSLVNRVKKSDTAASDAAATDAATTDTTTAN

SEQ ID NO: 9 - protein, 289 aa, Moraxella catarrhalis
CGQQTKEDINAQDTHSPKKLSVVTTFTVIADIAQNVAGEAADVQSITKAGAEIHEYEPTPQDVVKAQKADLILWNGLNLELWFEKFYHDTSNVPAVVVTQGITPINITEGAYKDMPNPHAWMSPSNALIYVENIKNALIKQDPANQEVYTKNAEQYSAKIKAMDAPLRAKLSQIPENQRWLVTSEGAFSYLANDYGLKEAYLWPINAEQQGSPQQVKSLIDTVRSNNIPVVFSESTISDKPAKQVAKETGAKYGGVLYVDSLSEAGGPVPTYLDLLQTTVSTIASGFEK

SEQ ID NO: 10 - DNA, 20 bp, Artificial Sequence
  Primer
tttaaataaaaagccatacg

SEQ ID NO: 11 - DNA, 30 bp, Artificial Sequence
  Primer
tagttagtcaaaattaacctaattgcttga

SEQ ID NO: 12 - DNA, 30 bp, Artificial Sequence
  Primer
aggttaattttgactaactaggaggaataa

SEQ ID NO: 13 - DNA, 30 bp, Artificial Sequence
  Primer
aaaaatataacattattccctccaggtact

SEQ ID NO: 14 - DNA, 30 bp, Artificial Sequence
  Primer
gggaataatgttatatttttaatatatttt

SEQ ID NO: 15 - DNA, 20 bp, Artificial Sequence
  Primer
cctgctggtgtcatgtatca

SEQ ID NO: 16 - DNA, 31 bp, Artificial Sequence
  Primer
gtacccatggaggtcagcagaccaaagaaga

SEQ ID NO: 17 - DNA, 30 bp, Artificial Sequence
  Primer
gatcggatcccttttcaaaaccgctggcga

SEQ ID NO: 18 - DNA, 20 bp, Artificial Sequence
  Primer
gcactcattaagcaagaccc

SEQ ID NO: 19 - DNA, 18 bp, Artificial Sequence
  Primer
gagccaaagcccttgcca

SEQ ID NO: 20 - DNA, 20 bp, Artificial Sequence
  Primer
ggcaagggctttggctcaag

SEQ ID NO: 21 - DNA, 19 bp, Artificial Sequence
  Primer
cagtcacttgatacatatc

SEQ ID NO: 22 - DNA, 19 bp, Artificial Sequence
  Primer
gatatgtatcaagtgactg

SEQ ID NO: 23 - DNA, 19 bp, Artificial Sequence
  Primer
caatcgtcgcacctgtcgc

SEQ ID NO: 24 - DNA, 20 bp, Artificial Sequence
  Primer
atgaaatcaatcaaaacttt

SEQ ID NO: 25 - DNA, 20 bp, Artificial Sequence
  Primer
tcacttttcaaaaccgctgg

(19) United States
(12) Patent Application Publication
(10) Pub. No.: US 2019/0343949 A1
(43) Pub. Date: Nov. 14, 2019

(54) VACCINE AGAINST MORAXELLA CATARRHALIS

(71) Applicant: The Research Foundation for The State University of New York, Amherst, NY (US)

(72) Inventors: Timothy F. MURPHY, East Amherst, NY (US); Aimee BRAUER, Niagara Falls, NY (US)

(21) Appl. No.: 16/482,648

(22) PCT Filed: Jan. 29, 2018
(86) PCT No.: PCT/US2018/015721

§ 371 Date: Jul. 31, 2019

Related U.S. Application Data
(60) Provisional application No. 62/452,408, filed on Jan. 31, 2017.

(51) Int. Cl. A61K 39/104; C07K 14/21

(52) U.S. Cl. CPC A61K 39/1045; C07K 14/212

(57) ABSTRACT

Immunogenic compositions comprising Moraxella catarrhalis protein AfeA, its variants and/or fragments are provided. Also provided are methods for use of these compositions for preventing or treating otitis media and exacerbations of chronic obstructive pulmonary disease (COPD).

### CROSS-REFERENCE TO RELATED APPLICATIONS

This application claims priority to U.S. Provisional application No. 62/452,408, filed on Jan. 31, 2017, the disclosure of which is incorporated herein by reference.

### BACKGROUND OF THE DISCLOSURE

Moraxella catarrhalis (M. catarrhalis) is a human respiratory tract pathogen that causes a substantial global burden of disease, particularly otitis media (middle ear infections) in children and respiratory tract infections (exacerbations) in adults with chronic obstructive pulmonary disease (COPD).

Approximately 80% of children experience an episode of otitis media by the age of three years and up to 30% of children experience recurrent otitis media, which is associated with delays in speech and language development as a result of impaired hearing (Otsuka et al., 2013, PLoS One 8:e68711; Vergison et al., 2010, Lancet Infect Dis 10:195-203; Teele et al., 1990, J Infect Dis 162:685-694). An estimated 709 million cases of otitis media occur annually worldwide (Monasta et al., 2012, PLoS One 7:e36226; Avnstorp et al., 2016, Int J Pediatr Otorhinolaryngol, 83:148-53). As the most common reason for infants and children to receive antibiotic therapy, otitis media is a main driver of the global crisis in antibiotic resistance in bacteria (Coker et al., 2010, JAMA 304:2161-9). A vaccine to prevent otitis media would have an enormous benefit in preventing global morbidity, reducing healthcare costs, and helping to ameliorate the growing problem of antibiotic resistance.

COPD is a debilitating disease that is the fourth most common cause of adult death in the US and the world (Decramer et al., 2012, Lancet 379:1341-51; Jemal et al., 2005, JAMA 294:1255-9). While death rates from heart disease and stroke are declining, the death rate from COPD has doubled since 1970 (Jemal et al., 2005, JAMA 294:1255-9). The course of COPD is characterized by intermittent worsening of symptoms called exacerbations. Approximately half of exacerbations are caused by bacterial infection (Sethi et al., 2008, N Engl J Med 359:2355-65). Exacerbations result in substantial morbidity and cost, including clinic visits, emergency room visits, hospital admissions, respiratory failure and death. Exacerbations accelerate decline in lung function (Hoogendoorn et al., 2010, Int J Chron Obstruct Pulmon Dis 5:435-44) and are the most important cause of the reduced quality of life in patients with COPD (Kessler et al., 2006, Chest 130:133-42). Remarkably, exacerbations of COPD that require hospital admission are associated with a 23% one-year mortality (Groenewegen et al., Chest 124:459-67; Soler-Cataluna et al., Thorax 60:925-31). Thus, one of the most urgent areas of research to impact patients with COPD is the development of approaches to preventing exacerbations.

To develop vaccines to prevent otitis media in children and exacerbations in adults with COPD, it is critical to know the etiology of these infections. The gold standard for determining the etiology of otitis media has been culture of middle ear fluid obtained by tympanocentesis. Based on middle ear fluid cultures, the three most common causes of otitis media are nontypeable (unencapsulated) Haemophilus influenzae, Streptococcus pneumoniae and M. catarrhalis.

M. catarrhalis has been overlooked as a pathogen in COPD because the organism is difficult to distinguish from commensal Neisseria, which are part of the normal flora of the human upper respiratory tract. M. catarrhalis is missed in sputum cultures by many clinical microbiology laboratories. Based on a rigorous prospective study using accurate methods to identify the organism, M. catarrhalis is considered to be the second most common cause of exacerbation of COPD after nontypeable H. influenzae. Adults with COPD experience 2 to 4 million exacerbations caused by M. catarrhalis annually in the US (Murphy et al., 2005, Am J Respir Crit Care Med 172:195-199).

As such there is an ongoing need to develop therapeutic and preventative approaches for conditions caused by M. catarrhalis in children and adults.

### SUMMARY OF THE DISCLOSURE

In this disclosure, we have identified and characterized a high value vaccine candidate antigen, AfeA protein of M. catarrhalis. Our data shows that AfeA has characteristics of an excellent vaccine antigen. For example, AfeA: 1) is highly conserved among strains, 2) induces high titer antibody that recognizes native protein following immunization with recombinant purified protein, 3) expresses abundant epitopes on the bacterial surface, 4) induces protective responses in the mouse pulmonary clearance model following aerosol challenge with M. catarrhalis, 5) is expressed during human respiratory tract infection and 6) binds ferric, ferrous, manganese and zinc ions.

In one aspect, this disclosure provides immunogenic compositions comprising AfeA, immunogenic variants thereof, or immunogenic fragments thereof. The immunogenic fragments comprise one or more surface exposed epitopes of AfeA in M. catarrhalis. The immunogenic compositions can, optionally, comprise one or more adjuvants. In a related aspect, the disclosure provides vaccine compositions comprising AfeA, immunogenic variants thereof or immunogenic fragments thereof, and one or more adjuvants. The compositions can be used for preventing or treating infections or diseases caused by M. catarrhalis in humans of all ages. For example, the compositions can be used as vaccines for preventing otitis media, particularly in children, and for preventing exacerbations of COPD, particularly in adults.

In one aspect, the disclosure provides methods for preventing or treating conditions caused by or exacerbated by M. catarrhalis. For example a method is provided for preventing or treating otitis media in an individual comprising administering to the individual a composition comprising an immunologically effective amount of AfeA, an immunogenic variant thereof or an immunogenic fragment thereof. In another example, a method is provided for preventing or treating exacerbations of COPD in an individual comprising administering to the individual a composition comprising an immunologically effective amount of AfeA, an immunogenic variant thereof, or an immunogenic fragment thereof. With respect to the prevention or treatment of otitis media, the individual can be of any age, but otitis media is known to occur in children, particularly age 3 and under, with much higher frequency than older children and adults. Similarly, with respect to prevention and treatment of exacerbations of COPD, the individual can be of any age, but such exacerbations are known to occur with higher frequency in adults.

In one aspect, this disclosure provides combination vaccines. The AfeA protein, an immunogenic variant thereof, or immunogenic fragments thereof may be combined with other immunogens in the treatment of otitis media, exacerbations of COPD or other infections or diseases that are caused by M. catarrhalis. In one embodiment, this disclosure provides a combination vaccine for the prevention or treatment of a disease or indication comprising AfeA protein, an immunogenic variant thereof, or immunogenic fragments thereof, and one or more immunogens from one or more microorganisms that may also cause the same disease or indication, and optionally, one or more adjuvants. For example, this disclosure provides a combination vaccine comprising AfeA protein, an immunogenic variant thereof, or immunogenic fragments thereof, and one or more immunogens from Haemophilus influenzae and/or Streptococcus pneumoniae. In one embodiment, the AfeA protein, immunogenic variants thereof, or immunogenic fragments thereof may be combined with immunogens that are directed to diseases or disorders other those caused by caused by M. catarrhalis and other than otitis media or exacerbations of COPD. For example, AfeA protein, immunogenic variants thereof, or immunogenic fragments thereof may be combined with childhood vaccine antigens such as diphtheria toxoid, tetanus toxoid, pertussis and the like.

### BRIEF DESCRIPTION OF THE FIGURES

FIG. 1. Schematic illustration of the afe gene cluster in the M. catarrhalis genome (A.) Arrows indicate direction of transcription and numbers indicate size of genes in base pairs (bp). Results of reverse transcriptase (RT) PCR with RNA extracted from M. catarrhalis 035E to detect afe gene cluster transcript in the regions that span genes as noted at the bottom (B.) Lanes: a, PCR product from genomic DNA template; lanes b. RT-PCR reaction in the absence of reverse transcriptase; lanes c, RT-PCR product; DNA standards are noted in kilobases. Bands in lanes c indicate that the gene cluster expresses a single transcript.

FIG. 2. Ethidium bromide-stained agarose gel showing amplicons of the afeA gene amplified from genomic DNA of 20 clinical isolates of M. catarrhalis. Lane a, 035E. Lanes b through k contain sputum isolates from adults experiencing exacerbations of COPD as follows: a, 6P29B1; b, 10P66B1; c, 14P30B1; d, 39P33B1; e, 47P31B1; f, M2, g, M3; h, M4; i, M5; j, M6. Lanes k through t contain middle ear fluid isolates obtained by tympanocentesis from children experiencing acute otitis media as follows: k, 2015; 1, 5193; m, 6955; n, 7169; o, 9483; p, 0701057VIL; q, 0701064V3L; r, 0702076SV4R; s, 0701062V1L; t, 0701067V3L. Molecular size markers are on the left in kilobases.

FIG. 3. Left Panel. Lane a: purified AfeA in Coomassie blue-stained sodium dodecyl (SDS) gel. Lane b: purified AfeA in silver stained SDS gel. Center Panel: immunoblot assay with rabbit antiserum to recombinant purified AfeA (1:10⁶ dilution). Right Panel immunoblot assay with rabbit antiserum to recombinant purified BCAA SBP1 (branched chain amino acid substrate binding protein 1). Lanes: c: whole cell lysate of wild type strain 035E, lanes d: whole cell lysate of afe knockout mutant, lanes e: whole cell lysate of afe complemented mutant. Molecular mass markers are shown in kilodaltons. Arrows denote AfeA.

FIG. 4. Immunoblot assay of whole cell lysates of 9 clinical isolates of M. catarrhalis probed with rabbit antiserum to recombinant purified AfeA (1:10⁶ dilution). Strains in lanes as follows: a, 6P29B1; b, 10P66B1; c, 14P30B1; d, 39P33B1; e, 47P31B1; f, M2, g, M3; h, M4; i, M5; Molecular mass markers are shown in kilodaltons on the left.

FIG. 5. Results of whole cell ELISA with M. catarrhalis strain 035E, afe knockout mutant, and complemented afe mutant coated onto wells and assayed with antisera as noted. X-axes are serum dilutions and Y-axes are optical density at 450 nm. Results are shown with pre-immune and immune antisera. A. AfeA antiserum with WT and afe knockout mutant. B. AfeA antiserum with complemented afe mutant. C. OppA antiserum with WT and afe knockout mutant (positive control—OppA is a surface protein). D. OppA antiserum with complemented afe mutant. E. BCAA antiserum with WT and afe knockout mutant (negative control—BCAA is a non surface protein). F. BCAA antiserum with complemented afe mutant. Error bars indicate the standard deviation of three independent experiments.

FIG. 6. Results of flow cytometry with M. catarrhalis wild-type (WT) 035E, afe knockout mutant and complemented afe mutant. X axes are fluorescence, and Y axes are cell counts. A. WT strain 035E, afe knockout mutant and complemented afe mutant assayed with AfeA antiserum (1:100) and preimmune serum (1:100). B. WT strain 035E and afeA knockout mutant assayed with OppA antiserum (1:100) and preimmune serum (1:100) (positive control—OppA is a surface protein). C. WT strain 035E and afeA knockout mutant assayed with BCAA antiserum (1:100) and preimmune serum (1:100) (negative control-BCAA is a non surface protein).

FIG. 7. Immunoblot assays with sera (1:2,000) pooled from mice immunized with PBS (negative control), purified recombinant AfeA (25 μg and 50 μg schedules as noted), and whole cells of M. catarrhalis 035E (A.) Lanes contain whole bacterial cell lysate of lane a, M. catarrhalis strain 035E and lane b, afe knockout mutant. Arrows denote AfeA. Molecular mass markers are shown in kilodaltons on the left. Results of pulmonary clearance three hours after aerosol challenge with M. catarrhalis 035E following immunization of groups of mice with PBS (negative control), recombinant AfeA, and whole cells of M. catarrhalis strain 035E (positive control) (B.) Y-axis is colony count (colony forming units/ml) in lung homogenates. Error bars represent the standard deviation (n=6). Statistically significant overall group differences were observed with a p-value of 0.0003. Results of pairwise comparisons with the PBS group (negative control) and associated p-values are shown.

FIG. 8. Results of ELISA to purified AfeA with 19 pairs of pre exacerbation and post exacerbation serum samples (1:4,000) from adults with COPD followed longitudinally. X-axis shows results from individual patients. Y-axis shows % change in optical density from pre exacerbation to post exacerbation value. Dotted line represents the cutoff for a significant change based on assays with control serum pairs.

FIG. 9. Sequence of AfeA. A) Nucleotide (SEQ ID NO:1); B) Protein (SEQ ID NO:2).

FIG. 10. Sequence of CysP. A) Nucleotide (SEQ ID NO:3); B) Protein (SEQ ID NO:4).

FIG. 11. Sequence of SBP2. A) Nucleotide (SEQ ID NO:5); B) Protein (SEQ ID NO:6).

FIG. 12. Sequence of OppA. A) Nucleotide (SEQ ID NO:7); B) Protein (SEQ ID NO:8).

### DESCRIPTION OF THE DISCLOSURE

Throughout this application, the use of the singular form encompasses the plural form and vice versa. For example, “a”, or “an” also includes a plurality of the referenced items, unless otherwise indicated.

Where a range of values is provided in this disclosure, it should be understood that each intervening value between the upper and lower limit of that range is also included, unless clearly indicated otherwise. The upper and lower limits from within the broad range may independently be included in the smaller ranges encompassed within the disclosure.

The terms “prevent” and “preventing” as used herein mean reducing the occurrence or recurrence of an infection, disease or disorder. It is not intended to be limited to complete prevention. In some cases, the onset may be delayed.

The terms “treatment” or “treating” as used herein mean alleviation of one or more of the symptoms or markers of the indication that is being treated. It is not intended to be limited to complete treatment.

The present disclosure provides immunogenic compositions comprising AfeA protein, immunogenic variants thereof, or immunogenic fragments thereof, and methods for using the compositions for prevention and/or treatment of infections or diseases caused by M. catarrhalis in children and adults. The AfeA protein (also referred to herein as a polypeptide) may be an isolated or purified protein. Isolated or purified protein may be obtained by methods known in the art, such as by isolation of the proteins from M. catarrhalis cultures, or by producing the proteins recombinantly from expression vectors inserted into cells, culturing the cells under conditions whereby the proteins are synthesized by the cells, and isolating the proteins from the cells using established procedures.

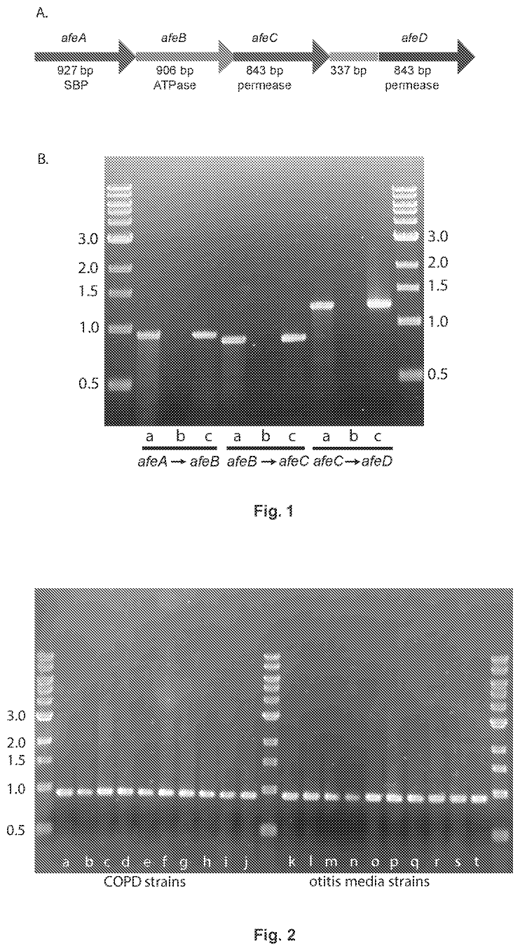

AfeA is an approximately 32 kDa protein. It is a substrate binding protein of an ATP binding cassette (ABC) transporter. ABC transporters generally include one or more permeases, ATPases and substrate binding proteins. AfeA is part of a gene cluster that includes genes that encode one substrate binding protein (AfeA), one ATPase (AfeB), and two permeases (AfeC and AfeD) (FIG. 1A). The complete amino acid sequence of AfeA is shown in SEQ ID NO:2. However, in the context of immunogenic compositions, a sequence of AfeA that is shorter than SEQ ID NO:2 can be used. For example, a polypeptide of SEQ ID NO:9, that does not have the signal peptide (i.e., does not have the first 19 amino acids of SEQ ID NO:2) can be used. In the generation of AfeA, the longer protein of SEQ ID NO:2 may be produced and then the signal peptide can be cleaved, or the shorter protein of SEQ ID NO:9 may be produced.

Substrate binding proteins (SBPs) of ABC transporter systems are located in the periplasm of Gram-negative bacteria and function to bind and transport ligands from the outer membrane to permeases in the cytoplasm for import (Maqbool et al., 2015, Biochem Soc Trans 43:1011-7; Wilkens et al., 2015, F1000Prime Rep 7:14). Based on homology with SBPs of other Gram-negative bacteria, AfeA is predicted to transport ferric ions and possibly other cations, including manganese and zinc. Data presented herein confirms that AfeA binds to ferrous and ferric ions, as well as manganese and zinc ions.

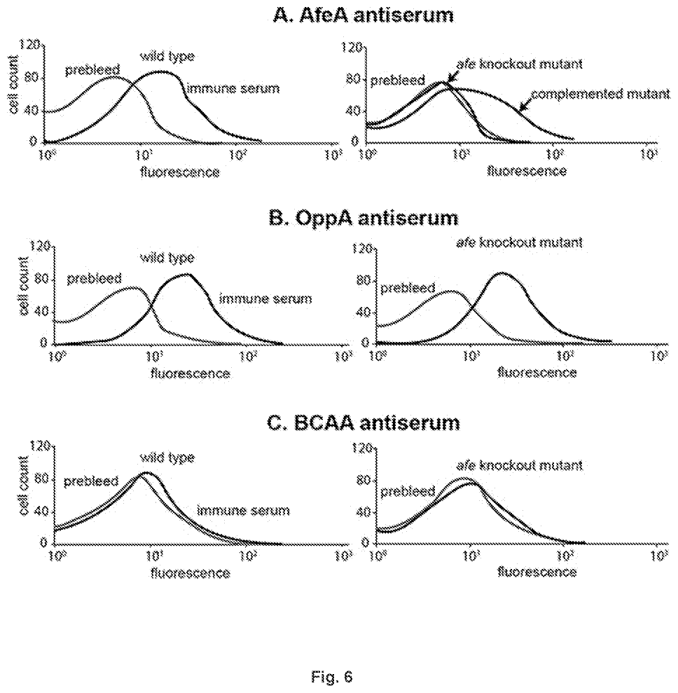

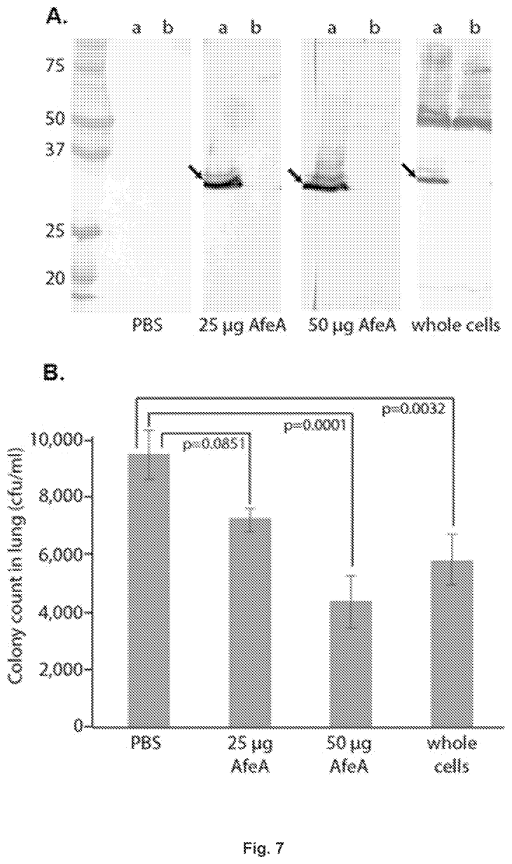

This disclosure provides data to demonstrate that AfeA exhibits surface epitopes. This finding was surprising. At least three independent lines of evidence presented in this disclosure support the conclusion that AfeA expresses epitopes on the bacterial surface: 1) whole cell ELISA with antiserum to AfeA (FIG. 5); 2) flow cytometry with antiserum to AfeA (FIG. 6); and 3) induction of protective immune responses by AfeA in the mouse pulmonary clearance model (FIG. 7). We further show that complementing the mutation restored surface exposure of AfeA, adding scientific rigor to our observation that AfeA has surface epitopes.

In one aspect, this disclosure provides immunogenic compositions comprising an effective amount of AfeA protein, an immunogenic variant thereof, or an immunogenic fragment thereof comprising at least one surface exposed epitope of AfeA. When a variant or fragment is recited to comprise a surface exposed epitope, it means that the variant or fragment comprises an epitope that, as part of AfeA protein in an intact M. catarrhalis is exposed on the surface of the bacteria (i.e., exposed such that is available for an immune response). In an embodiment, the variant or fragment comprises a plurality of surface exposed epitopes.

In one embodiment, this disclosure provides an immunogenic composition comprising a polypeptide AfeA having the sequence of SEQ ID NO:9. In one embodiment, the polypeptide is a variant and has a sequence that has at least 85% identity to the sequence of SEQ ID NO:9. The immunogenic compositions may comprise an adjuvant and are able to generate an immune response against M. catarrhalis. In one embodiment, the variant sequence may be shorter or longer than the reference sequence (such as shorter or longer by up to about 30 amino acids). In one embodiment, this disclosure provides an immunogenic composition comprising a fragment of AfeA and an adjuvant, said fragment comprising at least 15 contiguous amino acids from the sequence of SEQ ID NO:9 (shown below) or from a sequence having at least a 85% homology to the sequence of SEQ ID NO:9, and comprising at least one surface exposed epitope of AfeA. In one embodiment, the fragment is at least 15 amino acids long and has at least a 85% identity to a 15 amino acid long contiguous sequence from SEQ ID NO:9. For example, the fragments can be 15-30 amino acids long comprising 15-30 contiguous amino acids from the sequence of SEQ ID NO:9 or from a sequence having at least a 85% homology to the sequence of SEQ ID NO:9.

(SEQ ID NO: 9)
CGQQTKEDINAQDTHSPKKLSVVTTFTVIADIAQNVAGEAADVQSITKAG
AEIHEYEPTPQDVVKAQKADLILWNGLNLELWFEKFYHDTSNVPAVVVTQ
GITPINITEGAYKDMPNPHAWMSPSNALIYVENIKNALIKQDPANQEVYT
KNAEQYSAKIKAMDAPLRAKLSQIPENQRWLVTSEGAFSYLANDYGLKEA
YLWPINAEQQGSPQQVKSLIDTVRSNNIPVVFSESTISDKPAKQVAKETG
AKYGGVLYVDSLSEAGGPVPTYLDLLQTTVSTIASGFEK

The presence and/or identity of surface epitopes in the peptides or polypeptide fragments or variants can be determined by computational predictions based on predicted 3-D structure of the AfeA molecule or by determining if antibodies to selected peptides corresponding to fragments of the AfeA protein bind to the bacterial surface using whole cell ELISA or flow cytometry. For example, X-ray crystallography or NMR spectroscopy may be used to determine potential surface exposed epitopes. Alternatively, a common epitope mapping approach is the generation of consecutive overlapping synthetic peptides covering the entire primary sequence of a protein. Screening for antibodies can be done by ELISA or other immune based assays and ability to bind to surface epitopes can be confirmed on intact bacterial cells. Mapping of surface epitopes using cell-surface display systems can also be used (Hudson et al., Sci. Rep., 2012; 2:706, incorporated herein by reference).

The immunogenic compositions may be used as vaccines. The vaccine compositions are useful for preventing and/or treating infections and diseases caused by M. catarrhalis in individuals of any age. For example, vaccine compositions can be used for preventing, treating or ameliorating otitis media in children and adults, and exacerbations of COPD in adults. A vaccine to prevent or ameliorate the symptoms of M. catarrhalis infections has the potential to have a huge impact in preventing otitis media in children and infections in adults with COPD. AfeA protein, an immunogenic variant thereof, or a fragment thereof comprising at least one epitope may be used as an immunogen in vaccine formulations in the prevention of conditions which may occur with otitis media such as sinusitis and conjunctivitis, and which may occur with COPD such as other respiratory tract infections caused by M. catarrhalis. The proteins, or peptides described herein may be used as components of a fusion protein.

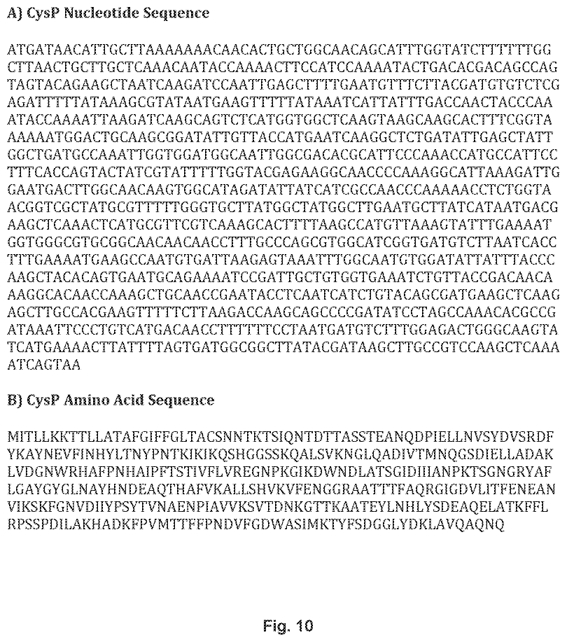

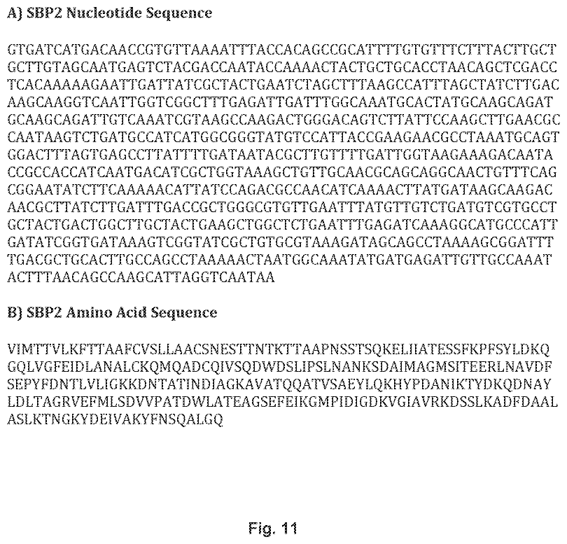

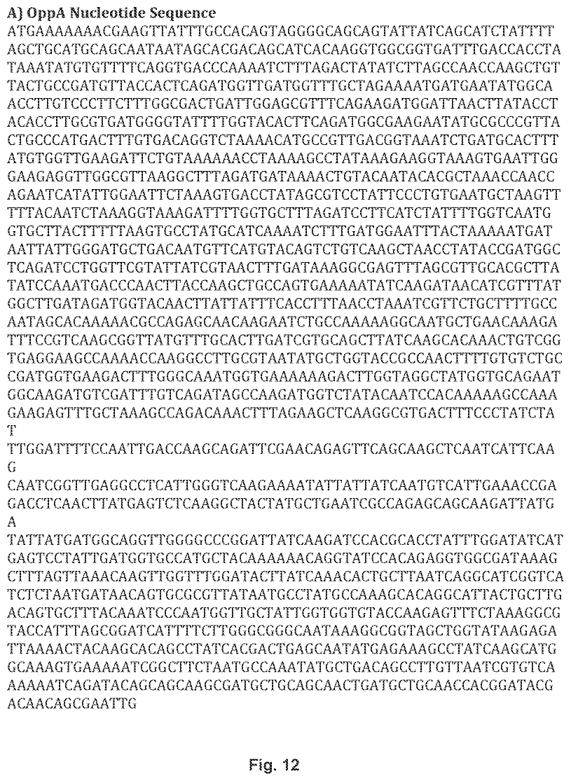

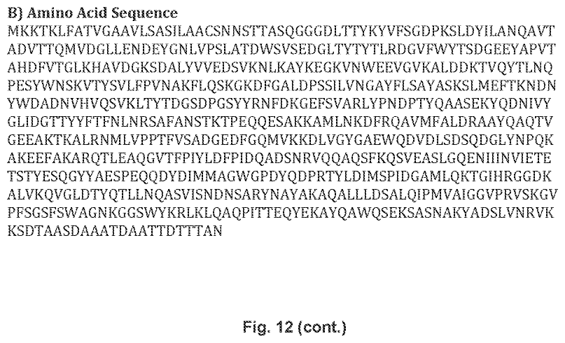

AfeA may be combined in a vaccine composition with other antigens against M. catarrhalis. One such antigen against M. catarrhalis is CysP or a fragment thereof comprising at least one epitope. The protein sequence of CysP is set forth in FIG. 10B. CysP can be made according to the protocol in (Murphy et al., Vaccine. 2016; 34(33):3855-61). Another antigen against M. catarrhalis is substrate binding protein SBP2 or a fragment thereof comprising at least one epitope. FIG. 11B provides the protein sequence of SBP2. Reference (Otsuka et al., Infect Immun. 2014; 82(8):3503-12) illustrates the manufacture of SBP2. Yet another antigen against M. catarrhalis is Oligopeptide Permease A (OppA) or a fragment thereof comprising at least one epitope. The protein sequence of OppA is depicted in FIG. 12B. A method of manufacturing OppA is detailed in reference (Yang et al., Infect Immun. 2011; 79(2):846-57) and U.S. Pat. No. 8,501,197). Still another antigen against M. catarrhalis is Outer Membrane Protein OMP CD or a fragment thereof comprising at least one epitope. U.S. Pat. No. 5,725,862 (“the '862 patent”) describes OMP CD. OMP CD, various fragments thereof comprising at least one epitope and nucleic acid sequences encoding OMP CD and its fragments are set forth in the '862 patent. Fragments of OMP CD comprising at least one epitope encoded by said nucleic acid sequences are within the scope of the inventive combination. The '862 patent also discusses how to make OMP CD, fragments thereof comprising at least one epitope and nucleic acids encoding said OMP CD and encoding said fragments.

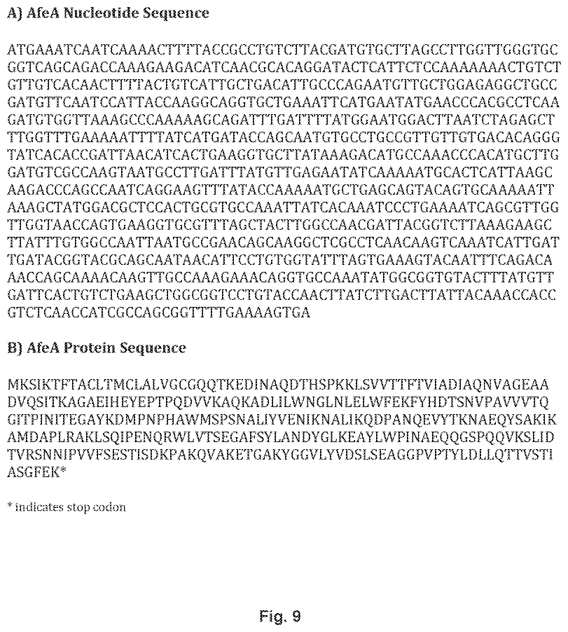

In the vaccines (including combination vaccines) and methods of this invention, AfeA protein or a fragment thereof comprising at least one epitope may be substituted by amino acid sequences sharing 80-99.9% homology, including all percentages and ranges therebetween, with the sequence of AfeA shown in FIG. 9B, which includes the signal peptide or with the sequence of AfeA in SEQ ID NO:9 in which the signal peptide (first 19 amino acids) are deleted, provided that the sequence containing the substitution comprises at least one epitope which can generate an immune response against Moraxella catarrhalis. For example, variants which are at least 85, 90, 95, or 99% identical to the sequence of AfeA disclosed herein may be used. Likewise, CysP, SBP2, OppA, OMP CD or a fragment of any of CysP, SBP2, OppA and OMP CD comprising at least one epitope may be substituted by amino acid sequences sharing 80-99.9% homology, including all percentages and ranges therebetween, with the sequences in FIGS. 10B, 11B, 12B and the '862 patent, respectively, provided that the sequence containing the substitution comprises at least one epitope which can generate an immune response against Moraxella catarrhalis. Variants which are at least 85, 90, 95 or 99% identical to the sequences of CysP, SBP2, OppA and OMP CD disclosed herein may be used The nucleic acid sequences described in the '862 patent which encode fragments of OMP CD comprising at least one epitope may also be modified to produce amino acid sequences sharing 80-99.9% homology with said OMP CD fragment, including all percentages and ranges therebetween, provided that the sequence containing the substitution comprises at least one epitope against Moraxella catarrhalis. The peptides and polypeptides used herein may also comprise purification tags, such as, poly histidine tags. Generally, that entails addition of at least 6 histidine residues at the N- or the C-terminus.

In an embodiment, the present disclosure provides combination vaccines which can provide protection or treatment against different pathogens. An effective vaccine strategy can be to prevent infections caused by M. catarrhalis and either or both of nontypeable H. influenzae and Streptococcus pneumoniae. Thus, in one embodiment, the disclosure provides a vaccine composition comprising an immunologically effective amount of AfeA protein or a fragment thereof comprising at least one surface exposed epitope in combination with an immunologically effective amount of at least one protein or fragment thereof from nontypeable (unencapsulated) Haemophilus influenzae and/or at least one protein or fragment thereof from S. pneumoniae.

In an embodiment, AfeA protein, immunogenic variants thereof, or immunogenic fragments thereof may be combined with other immunogens for the ease of administrations. For example, pediatric vaccines are often given as combination vaccines so as to reduce the number of injections a child has to receive. As such, in one embodiment, the present immunogens may be combined with other childhood vaccines that are commonly given, including one or more of diphtheria toxoid, tetanus toxoid, pertussis, and the like.

In an embodiment, the invention provides a vaccine composition comprising an immunologically effective amount of AfeA protein or a fragment thereof comprising at least one epitope in combination with an immunologically effective amount of any or all of the following: Pneumococcal 13-valent conjugate (e.g., Prevnar 13, Pfizer/Wyeth), Pneumococcal 7-valent conjugate (e.g., Prevnar, Pfizer/Wyeth) and 10-valent pneumococcal nontypeable Haemophilus influenzae protein D-conjugate vaccine (PHiD-CV) (e.g., Synflorix, GlaxoSmithKline). The inventive combination vaccines may include any or all of the excipients and adjuvants present in the relevant commercial formulations (e.g., Prevnar and/or Prevnar 13).

Using genetic engineering techniques, the inventive combination vaccines may incorporate into the same multivalent protein or peptide i) AfeA or at least one fragment thereof comprising at least one epitope and ii) at least one other antigen that is capable of inducing protective immunity (comprises at least one epitope) against nontypeable (unencapsulated) Haemophilus influenzae and/or Streptococcus pneumoniae and/or Moraxella catarrhalis. Exemplary antigens against M. catarrhalis other than AfeA or a fragment thereof include CysP, SBP2, OppA or OMP CD or a fragment of CysP, SBP2, OppA or OMP CD, wherein said fragment comprises at least one epitope. Alternatively, the AfeA or fragment thereof comprising at least one epitope and said other antigen(s) may be separate antigens within the vaccine composition.

The fragments for use in vaccine compositions in this disclosure can be peptides comprising 15-30 (and all inter numbers and ranges therebetween) or more contiguous amino acids from AfeA or other indicated proteins provided they are able to raise (either alone or collectively if a multiple fragments are used) a substantially similar immune response as compared to the native protein. In general, the fragments will contain one or more surface exposed epitopes in the native protein.

An adjuvant is typically used with the administration of immunogenic antigens. For example, an adjuvant can be used as a 0.001 to 50 wt % solution in phosphate buffered saline, and the antigen is present in the order of micrograms to milligrams, such as about 0.0001 to about 5 wt %, such as about 0.0001 to about 1 wt %, such as about 0.0001 to about 0.05 wt %. The antigen can be present in an amount in the order of micrograms to milligrams, or, about 0.001 to about 20 wt %, such as about 0.01 to about 10 wt %, or about 0.05 to about 5 wt %.

The adjuvant(s) may be a preferential inducer(s) of Th1 response. In one embodiment, the vaccine comprises aluminum phosphate as an adjuvant. In another embodiment, the adjuvant is alum (aluminum hydroxide and magnesium hydroxide, available from Thermo Scientific), MPL (monophosphorylated lipooligosaccharide), ASO4 (GlaxoSmithKline's 3-0-desacyl-4′-monophosphoryl lipid A adsorbed onto aluminum as hydroxide salt), or squalene such as MF59® (Novartis' microfluidised detergent stabilized oil-in-water emulsion comprising squalene and stabilized by Tween 80 and Span 85). Other examples of adjuvants include Adjuvant 65 (containing peanut oil, mannide monooleate and aluminum monostearate), oil emulsions, Ribi adjuvant, the pluronic polyols, polyamines, Avridine, Quil-A®, saponin, MPL, QS-21, liposomes, and mineral gels such as aluminum hydroxide. An example of an adjuvant that is considered to be a preferential inducer of Th1 response includes Monophosphoryl Lipid A or its derivatives (such as 3-de-O acylated monophosphoryl lipid A (3D-MPL).

The immunogenic or vaccine compositions may comprise a pharmaceutically acceptable carrier or excipient, which typically does not produce an adverse, allergic or undesirable reaction when administered to an individual, such as a human subject. Pharmaceutically acceptable carrier or excipient may be fillers (solids, liquids, semi-solids), diluents, encapsulating materials and the like. Examples include, but are not limited to, saline, buffered saline, dextrose, water, glycerol, ethanol etc.

The amount of the immunogen can be selected such that it produces an immunoprotective or therapeutic response and has minimal adverse side effects. One skilled in the art can select the amount of antigen required, but amounts generally can vary from 0.1 to 100 μg (and all values therebetween and ranges therebetween) for each administration. For example, the amount of AfeA per administration can be from 0.1 to 1, 1 to 5, 5 to 10, 10 to 25, 25 to 50, and 50 to 100 μg per administration. Depending on the formulation, one or more booster vaccinations may be used. The frequency of boosters may range from 1 month to 1 year.

In one embodiment, the vaccine is a suspension. In another embodiment, the vaccine is a colloidal dispersion, emulsion, cream, gel, ointment or solution. In a further embodiment, the vaccine comprises liposomes or nanoparticles.

Our data indicates AfeA has characteristics of an excellent vaccine antigen: 1) The afeA gene is present in all strains of M. catarrhalis; 2) It is highly conserved among clinical isolates that cause otitis media and infection in COPD; 3) AfeA expresses abundant epitopes on the bacterial surface that are accessible to potentially protective antibodies; 4) The protein is highly immunogenic (induces protective immune responses in the mouse following aerosol challenge with M. catarrhalis and induces antibody titers that detect the protein in dilutions in the millions following a standard immunization schedule; 5) AfeA is expressed during human infection, based on the development of antibody responses following exacerbations of COPD in selected patients; and 6) induces high titer antibody that recognizes native protein following immunization with recombinant purified protein.

The ability to invade and survive inside host cells is a potentially important virulence mechanism because intracellular M. catarrhalis serves as a reservoir for the bacterium to persist in the human respiratory tract (Heiniger et al., 2007, J Infect Dis 196:1080-7). A knockout mutant of the afe gene cluster has shown reduced growth rate in chemically defined media compared to wild type, and also showed reduced capacity for invasion of human respiratory epithelial cells (Murphy et al., 2016, PLoS One 11:e0158689). AfeA is likely a nutritional virulence factor that, as demonstrated herein, mediates the uptake of ferric ions, and possibly other cations, which are present in extremely low levels intracellularly. As such, a vaccine target that is also a virulence factor enhances its potential as a vaccine antigen because an immune response that targets a virulence factor may serve to inhibit infection in addition to mediating the binding of antibodies that mediate host responses to enhance clearance of the bacterium.

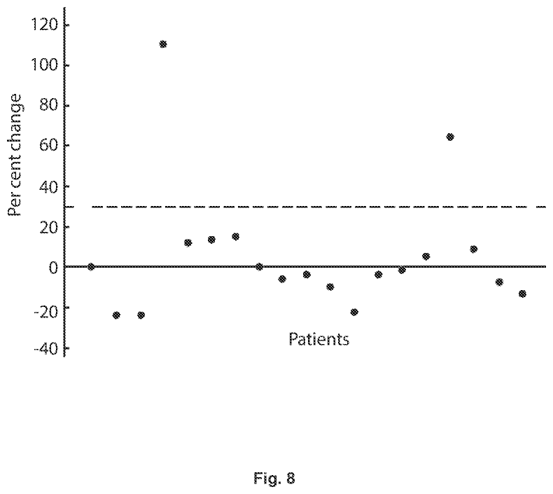

AfeA induces a new antibody response following exacerbations of COPD caused by M. catarrhalis (FIG. 8). The observation that AfeA induces a new antibody response in some patients indicates that the protein is expressed during human infection and is immunogenic among adults with COPD.

The disclosure also provides methods of immunizing a subject against Moraxella catarrhalis infections comprising administering an immunogenic composition or a vaccine composition according to the invention to the subject.

Many methods can be used for the introduction of a vaccine formulation into the human or animal to be vaccinated. The immunogenic compositions may be formulated for administration via systemic, dermal or mucosal routes. These include, but are not limited to, subcutaneous, transdermal, intradermal, intramuscular, intraperitoneal, intravenous, ocular, intranasal, oral, and by inhalation. The vaccine may further comprise a physiological carrier such as a polymer. In one embodiment, the vaccine is formulated for intramuscular administration. In other embodiments, the vaccine is formulated for intradermal, intraperitoneal, intravenous, subcutaneous, ocular, intranasal, mucosal, sublingual, buccal or oral administration. In an embodiment, the vaccine is formulated as an orally-disintegrating tablet (ODT).

Other uses of AfeA protein, a variant thereof, or a fragment thereof include use as immunogens for generating M. catarrhalis specific antisera which have therapeutic and/or diagnostic value. AfeA protein, a variant thereof, or a fragment thereof comprising at least one epitope thereof may be used to generate AfeA-specific antibodies which may be useful for passive immunization and as reagents for diagnostic assays directed to detecting the presence of M. catarrhalis in clinical specimens.

Purified AfeA protein, a variant thereof, or a fragment thereof comprising at least one surface exposed epitope thereof may be used as antigens in immunoassays for the detection of M. catarrhalis-specific antisera present in the body fluid of a subject suspected of having an infection caused by M. catarrhalis, e.g., by measuring an increase in serum titer of M. catarrhalis-specific antibody. The body fluids include, but are not limited to, middle ear fluid, sputum, blood, and fluids from the nasopharynx, eye, and adenoid. The detection of AfeA or a fragment thereof comprising at least one epitope as antigen(s) in immunoassays, includes any immunoassay known in the art including, but not limited to, radioimmunoassay, enzyme-linked immunosorbent assay (ELISA), “sandwich” assay, precipitin reaction, agglutination assay, fluorescent immunoassay, and chemiluminescence-based immunoassay.

The present disclosure also provides kits comprising one or more doses of the immunogenic formulations, and optionally, instructions for administration of the doses. The instructions may include a description of the composition and/or its use, guidance on storage of the compositions, route of administration, frequency and/or time of administration, any contraindications and the like. In addition to the immunogenic compositions and the instructions, the kit may include one or more devices for delivering the compositions. The device may depend upon the route of delivery. For example, the device may be a syringe (including multi-chambered syringes) for intramuscular delivery, a microneedle or set of microneedle arrays for transdermal delivery, a tiny balloon for intranasal delivery, or a small aerosol generating device for delivery by inhalation. The composition(s), the instructions, and the drug delivery device(s) may be packaged together.

The present disclosure provides compositions for administering to children (including infants) and adults for preventing, treating and/or ameliorating otitis media in children and adults. The invention further provides methods of immunizing a subject against otitis media comprising administering the present compositions to the subject, wherein the subject is a child or an adult. The term “child” or “children” as used herein is intended to include individuals of ages from newborn to up to 18 years. The term adult is intended to include individuals 18 and above. In one embodiment, the compositions are administered to the elderly (generally 60 or older, more typically, 65 or older).

Children under the age of about 6 are susceptible to otitis media. In one embodiment, vaccines comprising the compositions described herein can be administered to most or all children in a population. It is generally considered that for optimal effectiveness within a population, a vaccine for otitis media should be given to as many children as possible and preferably to all children. Vaccines can start at an early age after birth. For example, the present vaccine may be given at about 2 months of age, along with other routine childhood vaccines. The vaccine may be given to older children as well as adults. For children, the present compositions can be useful for mounting an initial immune response, while in adults, the compositions can act as boosters. Boosters may be administered to individuals at any age.

In one embodiment, the disclosure provides methods of preventing exacerbation of infections due to Moraxella catarrhalis in a subject comprising administering a vaccine according to the invention to the subject. In one embodiment, the infection is a respiratory tract infection. Thus, the invention provides methods of preventing exacerbation of a respiratory tract infection in a subject comprising administering a vaccine according to the invention to the subject. In one embodiment, the subject is afflicted with chronic obstructive pulmonary disease (COPD). The subject may be an adult.

The AfeA protein or a fragment thereof comprising at least one surface exposed epitope can be produced using recombinant DNA methods as illustrated herein, or can be synthesized chemically from the amino acid sequence disclosed in the present invention. Additionally, peptides can be produced from enzymatic or chemical cleavage of the mature protein. AfeA protein from M. catarrhalis or a fragment thereof comprising at least one epitope, or recombinant AfeA protein or a recombinant AfeA fragment comprising at least one epitope produced from an expression vector system can be purified with methods known in the art including detergent extraction, chromatography (e.g., ion exchange, affinity, immunoaffinity, or sizing columns), differential centrifugation, differential solubility, or other standard techniques for the purification of proteins.

In one embodiment, AfeA polypeptide and other polypeptides for use in vaccines of this disclosure can be made by adapting conventional molecular biology approaches. For example, DNA sequences encoding the polypeptides can be constructed based on the coding sequence of the polypeptides. Thus, the DNA sequences comprise a sequence encoding a contiguous polypeptide that includes the polypeptide that is used for stimulating an immune response. The resulting DNAs can be placed into any suitable expression vector. The expression vector can include any additional features that may or may not be part of the encoded polypeptides, such as any suitable promoter, restriction enzyme recognition sites, selectable markers, detectable markers, origins of replication, etc. The vectors can encode leader sequences, purification tags, and hinge segments that separate two or more other segments of the encoded protein. In an embodiment, a poly-Histidine tag can be encoded and thereby be incorporated into the polypeptide.

The expression vectors can be introduced into any suitable host cells, which can be prokaryotic and eukaryotic cells, including but not limited to E. coli cells, simian COS cells, Chinese Hamster Ovary (CHO) cells, human embryonic kidney 293 cells, or any other suitable cell types. The polypeptides can be expressed and separated from cell cultures that produce them using any suitable reagents and approaches, including but not necessarily limited to protein purification methods that use purification tags, including but not limited to histidine tags, and separating the polypeptides using such tags. Thus, the disclosure includes isolated polynucleotides encoding the polypeptides of this disclosure, cloning intermediates used to make such polynucleotides, expression vectors comprising the polynucleotides that encode the polypeptides, cells and cell cultures that comprise the DNA polynucleotides, cells and cell cultures that express the polypeptides, their progeny, cell culture media and cell lysates that contain the polypeptides, polypeptides that are separated from the cells and are optionally purified to any desirable degree of purity, and compositions comprising one or more polypeptides for use in vaccines.

One embodiment of the invention is directed to the construction of novel DNA sequences and vectors including plasmid DNA, and viral DNA such as from human viruses, animal viruses, insect viruses, or bacteriophages which can be used to direct the expression of AfeA protein or fragments thereof in appropriate host cells from which the expressed protein or fragments may be purified.

Another embodiment of the invention provides methods for molecular cloning of the gene encoding AfeA and nucleic acid sequences encoding an AfeA fragment comprising at least one epitope. The nucleic acid sequences disclosed herein can be used in molecular diagnostic assays for M. catarrhalis genetic material through nucleic acid hybridization, and including the synthesis of AfeA sequence-specific oligonucleotides for use as primers and/or probes in amplifying and detecting amplified nucleic acids.

According to one embodiment of the invention, recombinant DNA encoding AfeA or nucleic acid sequences encoding an AfeA fragment comprising at least one epitope is incorporated into an expression vector and the recombinant vector is introduced into an appropriate host cell thereby directing the expression of these sequences in that particular host cell. The expression system, comprising the recombinant vector introduced into the host cell, can be used (a) to produce AfeA protein or a fragment thereof comprising at least one epitope which can be purified for use as an immunogen in vaccine formulations; (b) to produce AfeA protein or a fragment thereof comprising at least one epitope to be used as an antigen for diagnostic immunoassays or for generating M. catarrhalis-specific antisera of therapeutic and/or diagnostic value; c) or if the recombinant expression vector is a live virus such as vaccinia virus, the vector itself may be used as a live or inactivated vaccine preparation to be introduced into the host's cells for expression of AfeA or a fragment thereof comprising at least one epitope; d) for introduction into live attenuated bacterial cells which are used to express AfeA protein or a fragment thereof comprising at least one epitope to vaccinate individuals; e) or for introduction directly into an individual to immunize against the encoded and expressed AfeA protein or a fragment thereof comprising at least one epitope. Furthermore, nucleotide sequences encoding AfeA or a fragment thereof comprising at least one epitope can be inserted into and expressed by various vectors including phage vectors and plasmids. Successful expression of the AfeA protein or fragment comprising at least one epitope thereof requires that either the insert comprising the gene or nucleic acid sequences, or the vector itself, contain the necessary elements for transcription and translation which is compatible with, and recognized by the particular host system used for expression. DNA encoding AfeA protein or a fragment thereof comprising at least one epitope can be synthesized or isolated and sequenced using the methods and primer sequences as illustrated herein. A variety of host systems may be utilized to express AfeA protein or a fragment thereof comprising at least one epitope, which include, but are not limited to bacteria transformed with a bacteriophage vector, plasmid vector, or cosmid DNA; yeast containing yeast vectors; fungi containing fungal vectors; insect cell lines infected with virus (e.g. baculovirus); and mammalian cell lines transfected with plasmid or viral expression vectors, or infected with recombinant virus (e.g. vaccinia virus, adenovirus, adeno-associated virus, retrovirus, etc.).

Using methods known in the art of molecular biology, various promoters and enhancers can be incorporated into the vector or the DNA sequence encoding AfeA amino acid sequences, i.e. recombinant AfeA, or a fragment thereof comprising at least one epitope, to increase the expression of the AfeA amino acid sequences, provided that the increased expression of the AfeA amino acid sequences is compatible with (for example, non-toxic to) the particular host cell system used. Thus, the DNA sequence can consist of the gene encoding AfeA protein, or any segment of the gene which encodes an epitope of the AfeA protein. Further, the DNA can be fused to DNA encoding other antigens, such as other M. catarrhalis antigens, S. pneumoniae antigens, nontypeable H. influenzae antigens or other bacterial, fungal, parasitic, or viral antigens to create a genetically fused (sharing a common peptide backbone) multivalent antigen for use as a vaccine composition.

The selection of the promoter will depend on the expression system used. Promoters vary in strength, i.e. ability to facilitate transcription. Generally, for the purpose of expressing a cloned gene, it is desirable to use a strong promoter in order to obtain a high level of transcription of the gene and expression into gene product. For example, bacterial, phage, or plasmid promoters known in the art from which a high level of transcription has been observed in a host cell system comprising E. coli include the lac promoter, trp promoter, recA promoter, ribosomal RNA promoter, the PR and PL promoters, lacUV5, ompF, bla, lpp, and the like, may be used to provide transcription of the inserted DNA sequence encoding AfeA amino acid sequences.

Additionally, if AfeA protein or a fragment thereof comprising at least one epitope may be lethal or detrimental to the host cells, the host cell strain/line and expression vectors may be chosen such that the action of the promoter is inhibited until specifically induced. For example, in certain operons the addition of specific inducers is necessary for efficient transcription of the inserted DNA (e.g., the lac operon is induced by the addition of lactose or isopropylthio-beta-D-galactoside). A variety of operons such as the trp operon, are under different control mechanisms. The trp operon is induced when tryptophan is absent in the growth media. The PL promoter can be induced by an increase in temperature of host cells containing a temperature sensitive lambda repressor. In this way, greater than 95% of the promoter-directed transcription may be inhibited in uninduced cells. Thus expression of recombinant AfeA protein or a fragment thereof comprising at least one epitope may be controlled by culturing transformed or transfected cells under conditions such that the promoter controlling the expression from the inserted DNA encoding AfeA amino acid sequences or a fragment thereof comprising at least one epitope is not induced, and when the cells reach a suitable density in the growth medium, the promoter can be induced for expression from the inserted DNA.

Other control elements for efficient gene transcription or message translation include enhancers, and regulatory signals. Enhancer sequences are DNA elements that appear to increase transcriptional efficiency in a manner relatively independent of their position and orientation with respect to a nearby gene. Thus, depending on the host cell expression vector system used, an enhancer may be placed either upstream or downstream from the inserted DNA sequences encoding AfeA amino acid sequences to increase transcriptional efficiency. These or other regulatory sites, such as transcription or translation initiation signals, can be used to regulate the expression of the gene encoding AfeA or nucleic acid sequences encoding AfeA fragments. Such regulatory elements may be inserted into DNA sequences encoding AfeA amino acid sequences or nearby vector DNA sequences using recombinant DNA methods described herein for insertion of DNA sequences.

Accordingly, M. catarrhalis nucleotide sequences containing regions encoding for AfeA protein or a fragment thereof comprising at least one epitope can be ligated into an expression vector at a specific site in relation to the vector's promoter, control, and regulatory elements so that when the recombinant vector is introduced into the host cell, the M. catarrhalis AfeA-specific DNA sequences can be expressed in the host cell. For example, the AfeA-specific DNA sequences containing its own regulatory elements can be ligated into an expression vector in a relation or orientation to the vector promoter, and control elements which will allow for expression of AfeA amino acid sequences. The recombinant vector is then introduced into the appropriate host cells, and the host cells are selected, and screened for those cells containing the recombinant vector. Selection and screening may be accomplished by methods known in the art including detecting the expression of a marker gene (e.g., drug resistance marker) present in the plasmid, immunoscreening for production of AfeA-specific epitopes using antisera generated to AfeA-specific epitopes, and probing the DNA of the host's cells for AfeA-specific nucleotide sequences using one or more oligonucleotides.

The AfeA protein or a fragment thereof comprising at least one epitope may be chemically synthesized, purified from M. catarrhalis or purified from a recombinant expression vector system. The gene encoding AfeA or nucleic acid sequences encoding a fragment thereof comprising at least one epitope may be incorporated into a bacterial or viral vaccine comprising recombinant bacteria or virus which is engineered to produce one or more epitopes of AfeA. Such hosts include, but are not limited to, bacterial transformants, yeast transformants, filamentous fungal transformants, and cultured cells that have been either infected or transfected with a vector which encodes AfeA amino acid sequences. Peptides or oligopeptides corresponding to portions of the AfeA protein may be produced from chemical or enzymatic cleavage of AfeA protein or chemically synthesized using methods known in the art and with the amino acid sequence deduced from the nucleotide sequence of the gene encoding AfeA as a reference.

In addition, the nucleic acid sequences encoding AfeA or a fragment thereof comprising at least one epitope, operatively linked to one or more regulatory elements, can be introduced directly into humans to express said AfeA or said fragment to elicit a protective immune response.

The AfeA protein or a fragment thereof comprising at least one epitope may be modified for ease of purification. In one embodiment, a 6-histidine tag is placed on the C-terminus for ease of purification. The 6-histidine tag is not present in the native protein or fragment.

Polypeptides for use in vaccines of this disclosure can be modified to improve certain biological properties, e.g., to improve stability, and/or to enhance certain capabilities, including but not necessarily limited to promoting T cell activation, and/or promoting interaction with phagocytes, such as macrophages and/or neutrophils. Other modifications may involve alteration of a glycosylation pattern, including deletions of one or more glycosylation sites, or addition of one or more glycosylation sites. The polypeptides of the vaccines may be provided in a composition, in a complex, or covalent linkage with other moieties. The polypeptides of the vaccines can be expressed as fusion proteins or can be chemically conjugated to other agents for numerous purposes, such as diagnostic applications. The polypeptides of the vaccines can accordingly be modified to be conjugated to detectable labels, including but not limited to visually detectable labels, such as compounds that can fluoresce or emit other detectable signals.

Modification of the AfeA protein or a fragment thereof comprising at least one epitope, such as by deletion and substitution of amino acids (and including extensions and additions to amino acids) and in other ways, may be made so as to not substantially detract from the immunological properties of the protein or fragment. In particular, the amino acid sequence may be altered by replacing one or more amino acids with functionally equivalent amino acids resulting in an alteration which is silent in terms of an observed difference in the physicochemical behavior of the protein or fragment. Functionally equivalent amino acids are known in the art as amino acids which are related and/or have similar polarity or charge. Thus, an amino acid sequence which is substantially that of the amino acid sequences described herein refers to an amino acid sequence that contains substitutions with functionally equivalent amino acids without changing the primary biological function of the protein or fragment.

Genetic engineering techniques may also be used to modify and/or adapt the encoded AfeA protein or a fragment thereof comprising at least one epitope. For example, site-directed mutagenesis to modify a protein fragment in regions outside the protective domains may be desirable to increase the solubility of the subfragment to allow for easier purification. Further, genetic engineering techniques can be used to generate DNA sequences encoding a portion of the amino acid sequence of AfeA. Restriction enzyme selection may be done so as not to destroy the immunopotency of the resultant AfeA protein or fragment thereof comprising at least one epitope. Antigenic sites of a protein may vary in size. A protein the size of AfeA contains many discrete antigenic sites. Therefore, partial gene sequences could encode antigenic epitopes of AfeA. Consequently, restriction enzyme combinations may be used to generate DNA sequences, which when inserted into the appropriate vector, are capable of directing the production of AfeA-specific amino acid sequences (protein or a fragment thereof) comprising one or more epitopes.

Throughout this application, various references are cited. These references, in their entireties, are hereby incorporated by reference.

The following example is provided to illustrate the invention and is not intended to be restrictive.

### Example 1

Identification and characterization of the afeA gene. As part of a genome mining approach to identify vaccine antigens of M. catarrhalis, we previously analyzed the genome of strain ATCC 43617 (accession numbers AX067426-AX067466) to identify open reading frames (ORFs) predicted to be exposed on the bacterial cell surface (Ruckdeschel et al., 2008, Infect Immun 76:1599-607). Of 348 ORFs predicted to be surface-exposed, 14 had homology to substrate binding proteins (SBPs) of ABC transporter systems. This observation led to an extensive evaluation of SBPs of ABC transporters of M. catarrhalis (Murphy et al., 2016, PLoS One 11:e0158689) and to the identification and characterization of three SBPs as promising vaccine antigens, including oligopeptide protein A (OppA), substrate binding protein 2 (SBP2) and CysP (Yang et al., 2011, Infect Immun 79:846-57; Otsuka et al., 2014, Infect Immun 82:3503-12; Murphy et al., 2016, Vaccine 19:34:3855-61).

Annotation of genes in strain BBH18 (GenBank NC_014147.1 GI:296112228) identified a gene cluster that has homology to genes that encode an ABC transporter system in Actinobacillus actinomycetemcomitans, afeABCD, that promotes cell growth under iron-chelated conditions (de Vries S P et al., 2010, J Bacteriol 192:3574-83; Rhodes et al., 2005, Infect Immun 73:3758-63). M. catarrhalis AfeA, the predicted SBP of this ABC transporter system, is 69% identical and 79% similar to the A. actinomycetemcomitans AfeA and is the subject of the current study. A Protein BLAST search with AfeA of M. catarrhalis (Protein ID WP_003658713.1) revealed that AfeA is 72 to 79% identical and 82 to 88% similar to predicted metal binding SBPs in other Moraxella species, Pasteurella species and Haemophilus species.

ABC transporters generally include one or more permeases, ATPases and substrate binding proteins. AfeA is part of a gene cluster that includes genes that encode one substrate binding protein (AfeA), one ATPase (AfeB), and two permeases (AfeC and AfeD) (FIG. 1A).

To determine whether the genes of the afeABCD gene cluster are transcribed as a single transcript or as multiple transcripts, reverse transcriptase PCR was performed using RNA isolated from M. catarrhalis strain 035E grown in broth using primers designed to correspond to transcripts that span adjacent genes in the gene cluster. Control assays lacking reverse transcriptase confirmed that the purified RNA was free of contaminating DNA (FIG. 1B, lanes b). FIG. 1B shows that the genes of the afeABCD gene cluster are transcribed as a single transcript (FIG. 1B lanes c).

Conservation of AfeA Among Strains of M. catarrhalis.

A tBLASTn search of the 3 publicly available complete genomes and 46 draft whole genome sequences available in GenBank with AfeA of M. catarrhalis strain BBH18 revealed that the afeA gene was present in the genomes of all 49 strains. A total of 21 strains showed 100% amino acid identity with AfeA; 22 strains showed 99% amino acid identity due to a single amino acid difference; one strain showed 98% identity (strain ctg3), and two strains showed 87% identity and 95% similarity (strains 304 and 324). The 3 remaining strains identified as M. catarrhalis showed lower homology. These strains are variant strains.

To assess the presence and conservation of the afeA gene in clinical isolates of M. catarrhalis, DNA purified from 20 clinical isolates was used as a template in a PCR reaction with primers that were designed to amplify the afeA gene (Table 2). The clinical isolates included 10 middle ear fluid isolates obtained by tympanocentesis from children with acute otitis media and 10 sputum isolates from adults who were experiencing exacerbations of COPD. A band of 927 bp with an identical size to that of strain 035E was detected in all 20 strains (FIG. 2). A negative control in which DNA template was replaced with water showed no band (data not shown). The sequences of the afeA amplicons revealed 99.7 to 100% identity in nucleotide sequence and 100% amino acid identity in all 20 strains. We conclude that the afeA gene is present in the genome of all clinical isolates of M. catarrhalis isolates tested to date and that the gene is highly conserved among strains.

Characterization of Purified Recombinant AfeA.

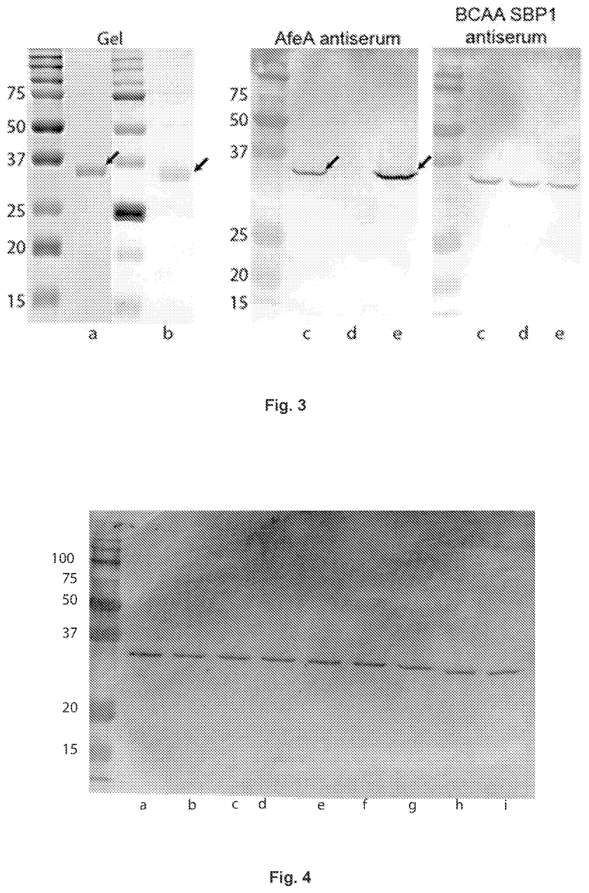

The M. catarrhalis afeA gene encodes a predicted lipoprotein with a 19 amino acid signal peptide at the amino terminus (LipoP 1.0). The mature AfeA protein, after cleavage of the signal peptide, contains 289 amino acids. The afeA gene region encoding the mature AfeA protein was inserted into the pCATCH vector to express recombinant AfeA as a lipoprotein with a C-terminal hexahistidine tag in E. coli BL21(DE3). Following expression and affinity purification with metal affinity resin, the purified protein separated as a single band of ˜32 kDa in SDS PAGE with Coomassie Blue stain and silver stain (FIG. 3).

Characterization of Afe Knockout Mutant.

An isogenic afe knockout mutant in strain 035E was constructed by replacing the afe gene cluster with a nonpolar kanamycin resistance cassette via homologous recombination. Antiserum raised to recombinant AfeA detected a single band of ˜32 kDa in immunoblot assay with a whole cell lysate of wild type strain 035E (FIG. 3, lanes c), confirming the specificity of the antiserum for AfeA. A whole cell lysate of the afe knockout mutant showed an absence of the ˜32 kDa band, confirming the absence of expression of AfeA in the mutant (FIG. 3, lanes d). The complemented afe mutant expressed AfeA as expected (FIG. 3, lanes e). The WT, afe knockout mutant and complemented mutant all expressed BCAA substrate binding protein, a control result indicating that expression of an unrelated substrate binding protein was unaffected by the genetic manipulations used to engineer and complement the afeA knockout mutant (FIG. 3, right panel).

Expression of AfeA by M. catarrhalis.

Rabbit antiserum raised to purified recombinant AfeA was used in immunoblot assay to assess expression of AfeA by M. catarrhalis. FIG. 3C shows that the antiserum recognizes a single band of the predicted size of AfeA (˜32 kDa) in a whole cell lysate of M. catarrhalis and detects no band in the knockout mutant. We conclude that antiserum raised to recombinant purified AfeA recognizes the AfeA protein expressed by M. catarrhalis.

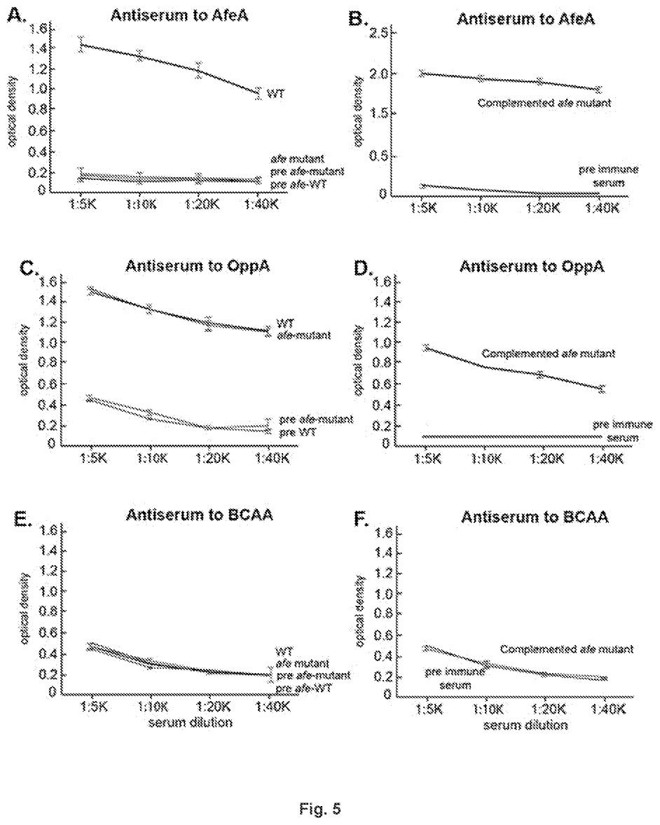

To assess the expression of AfeA by clinical isolates of M. catarrhalis, immunoblot assays with whole cell lysates of 20 clinical isolates (10 otitis media strains and 10 COPD exacerbation strains) were probed with AfeA antiserum. A band of ˜32 kDa was present in all 20 strains (FIG. 4). We conclude that clinical isolates of M. catarrhalis express AfeA during growth in vitro.

Expression of AfeA Epitopes on the Bacterial Surface.

Whole cell ELISAs were performed to determine the extent to which AfeA epitopes are expressed on the bacterial surface. Wells were coated with wild type (WT) M. catarrhalis 035E and the afe knockout mutant. Antiserum to OMP OppA was used as a positive control (surface protein) and antiserum to the BCAA SBP1 was used as a negative control (nonsurface protein). Anti AfeA antibody bound to the WT strain but not to the afe knockout mutant (FIG. 5A), while anti-OppA antibodies (positive control) bound to both strains (FIG. 5C), and anti-BCAA antibodies (negative control) bound to neither strain (FIG. 5E). The complemented afe mutant expressed AfeA on the bacterial surface; interestingly, the complemented afe mutant appears to express more surface exposed AfeA than WT, based on the higher OD observed with complemented mutant (FIG. 5B). The strong signal of antibodies binding to whole cells of the WT strain and the absence of binding of anti AfeA antibodies to the afe knockout mutant confirm the specificity of binding to AfeA in this assay and support the conclusion that AfeA expresses abundant epitopes on the bacterial cell surface.

As a second independent method to assess AfeA surface epitopes, we performed flow cytometry with WT afe knockout mutant and complemented mutant strains using antiserum to AfeA. Antibodies to AfeA demonstrate an increase in mean fluorescence intensity from pre immune to immune serum as indicated by a distinct shift of the curve to the right. (FIG. 6A). An assay of the same antiserum with the afe knockout mutant showed no shift, indicating that the antiserum contains AfeA-specific antibodies that bind to surface epitopes. Assay of the complemented afe showed partial restoration of activity (FIG. 6A, right). Antiserum to OppA (surface protein as positive control) showed binding to both wild type and afe mutant (FIG. 6B) and antiserum to BCAA (non surface protein as negative control) showed no binding to either strain (FIG. 6C). Based on the results of whole cell ELISA and flow cytometry, we conclude that M. catarrhalis expresses AfeA epitopes on the bacterial surface.

Induction of Mouse Pulmonary Clearance.

Immunoblot assays with pooled mouse serum showed that the mice immunized with AfeA developed antibodies to AfeA (FIG. 7A). To assess the effect of immunization with AfeA in the induction of protective immune responses, we assessed bacterial clearance three hours following aerosol challenge with M. catarrhalis in the mouse pulmonary clearance model. Mice immunized with purified AfeA (25 μg and 50 μg doses) showed enhanced clearance of M. catarrhalis from the lungs following aerosol challenge with M. catarrhalis compared to controls (FIG. 7B). Statistically significant overall group differences were observed with a p-value of 0.0003. Pairwise comparisons between PBS with 25 μg, 50 μg, and whole organism (positive control) had associated p-values of 0.0851, 0.0001, and 0.0032, respectively. A schedule using a 50 μg dose of AfeA induced a level of clearance similar to that observed with other vaccine antigens with this model (Otsuka et al., 2014, Infect Immun 82:3503-12; Murphy et al., 2016, Vaccine 19:34:3855-6; Forsgren et al., 2004, J Infect Dis 190:352-5; Liu et al., 2007, Infect Immun 75:2818-25; Murphy et al., 1998, J Infect Dis 178:1667-1675). The experiment depicted in FIG. 7B was repeated and yielded an identical result of enhanced clearance by approximately one half log of bacteria. We conclude that immunization of mice with AfeA induces protective responses in the mouse pulmonary challenge model.

Human Antibody Response to AfeA Following Infection.

To determine whether AfeA was expressed by M. catarrhalis during infection of the human respiratory tract, we performed ELISAs with serum samples from patients with COPD who experienced exacerbations caused by M. catarrhalis. The prospective study design enabled the use of pre exacerbation serum samples obtained 1 to 2 months before the patient acquired the infecting strain and post exacerbation samples from 1 to 2 months following infection. Two of 19 patients (10.5%) developed new antibody responses to AfeA following infection (FIG. 8). The observation that a subset of patients who experienced exacerbations of COPD developed new serum antibody responses indicates that AfeA is expressed in these patients during human infection.

Binding of Cations by AfeA.

Based on the observation that homologues of AfeA in other species are involved in metal transport, we performed experiments to test the hypothesis that AfeA binds cations by performing thermal shift assays. Initial experiments with purified, recombinant, lipidated AfeA failed to produce sharp melting temperatures (Tm), indicating that the protein did not form a stable conformation in the conditions of the assay, in spite of testing in several buffer conditions. Therefore, we engineered a construct that expressed non lipidated AfeA and purified the protein using buffers to which a cation chelating resin (Chelex® 100 (Sigma)), has been added. This approach resulted in sharp, biphasic melting curves, indicating that the protein formed stable conformations and some of the protein was bound by a ligand while some was not (Table 1).

Addition of selected metals resulted in thermal shift of the curve from a lower Tm (corresponding to unbound AfeA) to a curve with an increased Tm indicating binding of the added ligand to AfeA (Table 1). Specific binding of ferric, ferrous, manganese and zinc, was observed with a thermal shift of ˜23° C. with each of these cations. No thermal shift was observed with magnesium ions, confirming the specificity of binding in the assay. We conclude that AfeA binds ferric, ferrous, manganese and zinc ions.

TABLE 1
Melting temperatures and results of thermal shift
assays with purified non-lipidated recombinant AfeA

| Sample | Cation | Tₘ (° C.)^{a} | Δ Tₘ (° C.)^{b} |
|---|---|---|---|
| Non lipidated AfeA alone | — | 53.1, 76.7 | — |
| +1 mM MgCl₂ | Mg⁺⁺ | 53.6, 76.9 | 0.5 |
| +1 mM MnCl₂ | Mn⁺⁺ | 76.4 | 23.3 |
| +1 mM ZnCl₂ | Zn⁺⁺ | 77.1 | 24.0 |
| +1 mM Fe(NO3)₃ | Fe⁺⁺⁺ | 76.8 | 23.7 |
| +1 mM FeCl₃ | Fe⁺⁺⁺ | 76.7 | 23.6 |
| +1 mM FeCl₂ | Fe⁺⁺ | 76.7 | 23.6 |

^{a}Tₘ (° C.), melting temperature
^{b}Tₘ (° C.), thermal shift

Methods

Bacterial Strains and Growth.

M. catarrhalis strain 035E was provided by Eric Hansen. Strains 2015, 5193, 6955, 7169, 9483, 0701057VIL, 0701064V3L, 0702076SV4R, 0701062V1L and 0701067V3L are middle ear fluid isolates obtained via tympanocentesis from children with otitis media provided by Howard Faden in Buffalo N.Y. and Janet Casey in Rochester N.Y. Strains 6P29B1, 10P66B1, 14P30B1, 39P33B1 and 47P31B1 are sputum isolates obtained from adults with COPD during exacerbations as part of a prospective study. Strains M2, M3, M4, M5 and M6 are sputum isolates from adults with COPD provided by Daniel Musher in Houston Tex. Pulsed-field gel electrophoresis of genomic DNA cut with SmaI showed that the strains are genetically diverse. M. catarrhalis strains were grown on brain heart infusion (BHI) plates at 37° C. with 5% CO₂ or in BHI broth with shaking at 37° C.

Chemically competent Escherichia coli strains Top 10 and BL21(DE3) were obtained from Invitrogen (Carlsbad, Calif.) and were grown at 37° C. on Luria-Bertani (LB) plates or in LB broth.

Genomic DNA and RNA Purification.

Genomic DNA of M. catarrhalis strains was purified with the Wizard genomic DNA purification kit (Promega, Madison, Wis.) according to the manufacturer's instructions.

RNA from strain 035E was isolated with the Qiagen RNeasy minikit (Qiagen, Valencia, Calif.) and DNA contamination was removed with the RQ1 RNase-Free DNase kit (Promega).

Reverse Transcriptase PCR (RT-PCR).

RT-PCR was performed with the Qiagen One-Step RT-PCR kit according to the manufacturer's instructions, with 50 ng of RNA per reaction mixture.

Cloning the afeA Gene.

The afeA gene from M. catarrhalis strain 035E was cloned into the plasmid pCATCH, which allows for expression of recombinant lipoproteins in E. coli, using previously described methods (Yang et al., 2011, Infect Immun 79:846-57; Murphy et al., 2016, Vaccine 19:34:3855-61, Ruckdeschel et al., 2009, Vaccine 27:7065-72). Oligonucleotide primers corresponding to the 5′ end starting after the predicted cysteine codon and the 3′ end of the genes were designed with NcoI and BamH1 restriction sites (Table 2). The genes were amplified by PCR from genomic DNA of M. catarrhalis strain 035E. The resultant PCR product was ligated into pCATCH and transformed into E. coli TOP10 cells. Colonies were picked, grown in broth, and plasmids were purified. PCR and sequencing confirmed the insertion of the gene into the plasmid called pAfeA.

TABLE 2
Primer sequences

| Name of primer | Experiment | Sequence |
|---|---|---|
| Afe frag1 F1 | Mutant fragment 1 | TTTAAATAAAAAGCCATACG |
| | | (SEQ ID NO: 10) |
| Afe frag1 R1 | Mutant fragment 1 | TAGTTAGTCAAAATTAACCTAATTGCTTGA |
| | | (SEQ ID NO: 11) |
| Afe frag2 F1 | Mutant fragment 2 | AGGTTAATTTTGACTAACTAGGAGGAATAA |
| | | (SEQ ID NO: 12) |
| Afe frag2 R1 | Mutant fragment 2 | AAAAATATAACATTATTCCCTCCAGGTACT |
| | | (SEQ ID NO: 13) |
| Afe frag3 F1 | Mutant fragment 3 | GGGAATAATGTTATATTTTTAATATATTTT |
| | | (SEQ ID NO: 14) |
| Afe frag3 R1 | Mutant fragment 3 | CCTGCTGGTGTCATGTATCA |
| | | (SEQ ID NO: 15) |
| Afe lipo-protein | Clone AfeA gene | GTACCCATGGAGGTCAGCAGACCAAAGAAGA |
| F1 | | (SEQ ID NO: 16) |
| Afe lipo-protein | Clone AfeA gene | GATCGGATCCCTTTTCAAAACCGCTGGCGA |
| R1 | | (SEQ ID NO: 17) |
| AfeA 5 | RT PCR afeA-afeB | GCACTCATTAAGCAAGACCC |
| | | (SEQ ID NO: 18) |
| AfeB 3 | RT PCR afeA-afeB | GAGCCAAAGCCCTTGCCA |
| | | (SEQ ID NO: 19) |
| AfeB 5 | RT-PCR afeB- | GGCAAGGGCTTTGGCTCAAG |
| | afeC | (SEQ ID NO: 20) |
| AfeC 3 | RT-PCR afeB- | CAGTCACTTGATACATATC |
| | afeC | (SEQ ID NO: 21) |
| AfeC 5 | RT-PCR afeC- | GATATGTATCAAGTGACTG |
| | afeD | (SEQ ID NO: 22) |
| AfeD 3 | RT-PCR afeC- | CAATCGTCGCACCTGTCGC |
| | afeD | (SEQ ID NO: 23) |
| AfeA F1 | Amplify afeA | ATGAAATCAATCAAAACTTT |
| | | (SEQ ID NO: 24) |
| AfeA R1 | Amplify afeA | TCACTTTTCAAAACCGCTGG |
| | | (SEQ ID NO: 25) |

Expression and Purification of Recombinant AfeA.

Following growth of 50 ml of culture in LB broth with 50 μg kanamycin to an OD₆₀₀ of 0.6, AfeA expression was induced with 4 mM IPTG (isopropyl-ß-D-thiogalactopyranoside) for 4 hours at 37° C. The bacteria were then harvested by centrifugation at 4,000 g for 15 minutes at 4° C. The pellet was suspended in 10 ml of lysis buffer (0.020 M sodium phosphate, 0.5 M NaCl, 1 mg/ml lysozyme, 1× Protease Arrest™ (protease inhibitor), pH 7.4) and mixed with a nutator for 30 minutes at room temperature. The suspension was then sonicated on ice with a sonicator (Branson Sonifier® 450) at setting 6, using an 80% pulsed cycle of four 30-second bursts with 2-minute pauses. The sonicated bacterial lysate was centrifuged at 10,000 g for 20 minutes at 4° C. The pellet was suspended in 5 ml of Urea Lysis Buffer containing 0.05 M NaH₂PO₄, 0.01 M tris [pH 8], 6 M urea, 0.1 M NaCl, pH7.5 plus 25 μl of Protease Arrest and mixed on a nutator for 20 minutes until the lysate became clear.

AfeA was purified by affinity chromatography using BD talon resin (BD Biosciences, Palo Alto Calif.) through the 6-histidine tag, which is on the carboxy terminus of the recombinant AfeA, using a modification of previously described methods (Yang et al., 2011, Infect Immun 79:846-57; Otsuka et al., 2014, Infect Immun 82:3503-12). An aliquot of 2 ml of BD talon resin was centrifuged at 750 g for 5 minutes at 4° C., suspended in Urea Lysis Buffer, incubated for 10 minutes and centrifuged again. The resin was suspended in 5 ml of cleared bacterial lysate and mixed by nutation for 30 minutes at room temperature. The suspension was centrifuged at 750 g for 5 minutes at 4° C. The resin, containing bound protein was washed in Urea Lysis Buffer twice for 10 minutes. To elute recombinant protein, the washed resin was suspended in 1 ml of the same buffer containing 0.15 M imidazole and mixed by nutation for 10 minutes at room temperature. The resin was removed by centrifugation and the supernatant containing purified recombinant protein was collected. The elution was repeated and the eluates were pooled. AfeA was refolded by sequential dialysis in buffers that contained decreasing concentrations of arginine (0.5 M to 0.005 M). Protein concentration was measured by the method of Lowry (Sigma). The purity of the protein was assessed by sodium dodecyl sulfate polyacrylamide gel electrophoresis (SDS PAGE) and Coomassie Blue staining.

To express and purify recombinant AfeA that lacks the amino terminal linked lipoprotein for thermal shift assays, the afeA gene encoding the mature AfeA protein was amplified by PCR from genomic DNA of strain 035E using primers noted in Table 2 and ligated into plasmid pET 101 D-TOPO (Invitrogen). The ligation mixture was transformed into the chemically competent E. coli strain Top 10 and grown on BHI plates containing 50 μg/ml carbenicillin. The AfeA protein was expressed as described above and the recombinant protein was found in the supernatant following sonication. Nonlipidated recombinant AfeA was purified from the supernatant using the same method as for the recombinant lipidated AfeA protein described above.

Development of Antiserum to AfeA.

To develop antiserum to AfeA, purified recombinant AfeA was sent to Covance (Denver, Pa.) for antibody production in New Zealand White rabbits using a 59-day protocol. Briefly, 250 μg purified AfeA was emulsified 1:1 in complete Freund's adjuvant for initial subcutaneous immunization. Subsequent immunization followed a 3-week cycle of boosts with 125 μg AfeA emulsified 1:1 in incomplete Freund's adjuvant. Serum was collected 2 weeks after the second boost.

Construction of Afe Knockout Mutant.

A knockout mutant in which the entire gene cluster in which the afeA gene is located was engineered by using overlap extension PCR and homologous recombination as we have described previously with several M. catarrhalis genes (Murphy et al., 2016, PLoS One 11:e0158689; Yang et al., 2011, Infect Immun 79:846-57; Otsuka et al., 2014, Infect Immun 82:3503-12). Briefly, the transforming DNA for the mutant was composed of 3 overlapping fragments that included ˜1 kb upstream of the afe gene cluster (fragment 1), the nonpolar kanamycin resistance cassette amplified from plasmid pUC18K (fragment 2), and ˜1 kb downstream of the gene cluster. A mutant was constructed by transformation of M. catarrhalis strain 035E with a fragment composed of fragments 1, 2, and 3 and selection on brain heart infusion (BHI) plates containing 50 μg/ml of kanamycin. The insert and surrounding sequences of the mutant were confirmed by sequence analysis.

Complementation of the Afe Mutant.

Complementation was accomplished with plasmid pWW115 using previously described methods (Wang et al., 2006, Plasmid 56:133-7; Jones et al., 2014, Infect Immun 82:4758-66). Briefly, a fragment containing the afe gene cluster and 300 bp upstream to include the promoter of the afe operon and 300 bp downstream was amplified from genomic DNA of strain 035E and ligated into pWW115 using primers that included a BamHI site and a SacI site (Table 2). After confirming the insert sequence of the resulting plasmid construct, the afe mutant was transformed with the plasmid onto a BHI agar plate inoculated with 100 μl of 035E at an OD₆₀₀ of 0.2 and incubated for 5 h at 37° C. Spots were then spread onto BHI agar plates that contained 100 μg of spectinomycin and incubated overnight. The resulting colonies were picked and the afe operon and surrounding regions were confirmed with sequencing and immunoblot assay with antibody to AfeA. This complemented mutant was grown in the presence of spectinomycin for all experiments.

Whole Cell ELISA.

To assess binding of antibodies to epitopes on the bacterial surface, whole-cell enzyme-linked immunosorbent assay (ELISA) was performed. M. catarrhalis wild type strain 035E and the corresponding afe knockout mutant were grown in BHI broth to an OD₆₀₀ of 0.2, harvested by centrifugation, and resuspended in PBS. A volume of 100 μl of the suspension was added to each well of a 96-well microtiter Immunolon 4 plate (Thermo Labsystems, Franklin, Mass.) and incubated overnight at 4° C. to coat the wells with bacterial cells. Wells with PBS alone were included as controls. Wells were washed once with 0.05% Tween 20 in phosphate buffered saline (PBST) and blocked with 3% nonfat dry milk in PBS for 1 hour at room temperature, after which wells were washed 3 times with PBST. Paired rabbit antisera (pre-immune and immune) were diluted 1:5000, 1:10,000, 1:20,000 and 1:40,000 in diluent buffer (1% nonfat dry milk in PBST) and added to the sham-coated control wells and whole bacterial cell sample wells in parallel. After incubation for 2 hours at 37° C., wells were washed 3 times with PBST and a 1:3,000 dilution of peroxidase-labeled secondary antibody, anti-rabbit IgG (KPL, Gaithersburg, Md.) diluted in PBST plus 3% heat-inactivated goat serum was added. After another 1 hour of incubation at room temperature, wells were washed 3 times with PBST and color developing reagent was added. The reaction was allowed to proceed for 15 minutes and was stopped with 2 M sulfuric acid. The absorbance at 450 nm was determined using a Bio-Rad model 3550-UV microplate reader (Hercules, Calif.).

Mouse Pulmonary Clearance Model.

All animal studies were reviewed and approved by the University at Buffalo Institutional Animal Care and Use Committee. Systemic immunization was accomplished with groups of 6 Balb/c mice that were immunized subcutaneously with 25 μg or 50 μg of purified recombinant AfeA emulsified in incomplete Freund's adjuvant. Additional controls were immunized with either PBS plus adjuvant (negative control, n=6) or formalin-killed M. catarrhalis 035E emulsified in incomplete Freund's adjuvant (positive control, n=6). Injections were repeated at 14 and 28 days after the initial immunization. Mice were challenged on day 35 as described below.

Although, M. catarrhalis, an exclusive human pathogen, does not persistently colonize or cause infection in experimental animals, the induction of a protective response in an animal model by a surface exposed molecule is predictive of an effective vaccine antigen. The animal model used in the present disclosure is a mouse pulmonary clearance model for M. catarrhalis and it measures the rate of clearance of bacteria from the lungs. The model is quantitative, reproducible, used by several research groups and is the most widely used model to assess vaccine antigens of M. catarrhalis (Yang et al., 2011, Infect Immun 79:846-57; Otsuka et al., 2014, Infect Immun 82:3503-12; Adlowitz et al., 2006, FEMS Immunol Med Microbiol 46:139-46; Prymula et al., 2006, Lancet 367:740-8).

To determine if immunization with AfeA induces potentially protective responses in vivo, the mouse pulmonary clearance model was performed as described previously. An overnight culture of M. catarrhalis 035E was inoculated into 100 ml BHI broth to an OD₆₀₀ of ˜0.05 and grown to an OD₆₀₀ of ˜0.3. Bacteria were collected by centrifugation and resuspended in 10 ml PCGM buffer (4.3 mM NaHPO₄, 1.4 mM KH₂ PO₄, 137 mM NaCl, 2.7 mM KCl, 5 mM CaCl₂, 0.5 mM MgCl2, 0.1% gelatin, pH 7.3). An aliquot of suspension was diluted and plated to determine the starting concentration of bacteria. Ten ml of the bacterial suspension (˜10⁹ cfu/ml) was placed in the nebulizer of a Glas-Col Inhalational Exposure System model 099C A4212 (Glas-Col, Terre Haute, Ind.). Mice were challenged using this inhalation system with the following settings: 10 min preheat, 40 min nebulization, 30 min cloud decay, 10 min decontamination, vacuum flow meter at 60 cubic feet/h, compressed air flow meter at 10 cubic feet/h. With this system, all mice are challenged simultaneously with the identical number of bacteria.

Three hours post-challenge, the mice were euthanized by inhalation of isoflurane. Lungs were then harvested and homogenized on ice in 5 ml PCGM buffer using a tissue homogenizer. Aliquots of 50 μl of undiluted and 1:10 diluted lung homogenate were plated in duplicate and incubated at 35° C. in 5% CO₂ overnight. Colonies were counted the following day.

The statistical assessment of colony counts was based on a standard analysis of variance (ANOVA) model. In addition to testing for overall group differences, pairwise comparisons between PBS and each of the other three groups were made in conjunction with Dunnett's adjustment for multiple comparisons performed at a 0.05 family-wise error rate. Standard diagnostic plots were used to assess model fit with no violations of model assumptions observed. Analyses were carried out using SAS version 9.4 statistical software (Cary, N.C.).

ELISA with Human Serum Samples.

Serum samples were obtained from adults with COPD who were part of a 20-year prospective, observational study conducted at the Buffalo Veterans Affairs Medical Center that has been described previously (Murphy et al., 2005, Am J Respir Crit Care Med 172:195-199; Sethi et al., 2002, N Engl J Med 347:465-471). The study was approved by the Veterans Affairs Western New York Healthcare System Human Studies Subcommittee and the University at Buffalo Institutional Review Board. Patients with COPD were seen monthly and at times when an exacerbation was suspected. At each visit expectorated sputum samples and blood samples were collected and clinical criteria were used to determine whether patients were experiencing an exacerbation or whether they were clinically stable. An exacerbation strain was defined as a strain of M. catarrhalis that was isolated from sputum and that was acquired simultaneous with the onset of symptoms of an exacerbation using previously described methods (Sethi et al., 2002, N Engl J Med 347:465-471). Nineteen paired pre exacerbation serum samples were obtained 1 to 2 months prior to exacerbation and post exacerbation serum samples were obtained 1 to 2 months following the same exacerbation. The serum samples were used to analyze the human antibody response to purified recombinant AfeA.

ELISAs were performed using previously described methods (Ruckdeschel et al., 2008, Infect Immun 76:1599-607). Wells were coated with 01 μg/ml of purified AfeA and serum samples were assayed at dilutions of 1:4000. These conditions were determined to yield a linear curve between the OD₄₅₀ and serum dilution in preliminary experiments. The pre and post exacerbation serum pairs were always tested in the same assay on the same plate. The percent change in antibody level from the pre exacerbation to post exacerbation serum samples was calculated with the following formula: [(OD of post exacerbation sample−OD of pre exacerbation sample)/OD of pre exacerbation sample]×100. A cutoff of 30% for a significant change between pre and post exacerbation was set based on control assays performed in previous studies of 6 proteins (Ruckdeschel et al., 2008, Infect Immun 76:1599-607; Yang et al., 2011, Infect Immun 79:846-57; Adlowitz et al., 2005, Infect Immun 73:6601-7; Adlowitz et al., FEMS Immunol Med Microbiol 46:139-46). In these assays, paired control serum samples obtained 2 months apart (the same time interval used for the experimental samples) from COPD patients whose sputum cultures were negative for M. catarrhalis and who were clinically stable and free of exacerbation were assayed and used to determine the cutoff value.

Thermal Shift Assay.

Thermal shift assays were performed using a Stratagene Mx3005P real-time PCR instrument (Stratagene, La Jolla, Calif.) as previously described (Murphy et al., 2016, Vaccine 19:34:3855-61; Koszelak-Rosenblum et al., 2008, J Biol Chem 283:24962-71; Otsuka et al., 2015, Infect Immun 84:432-8). Briefly, purified, recombinant, nonlipidated AfeA was studied at a concentration of 10 μg in a 30-μl volume in buffer (0.01 M tris, 0.15 NaCl, pH 7.4) to which metal salts were added to a final concentration of 1 mM. SYPRO Orange (Sigma) was added as a fluorescence reporter at a 1:1,000 dilution from its stock solution. The change in fluorescence was monitored using a Cy3 filter, with excitation and emission wavelengths of 545 nM and 568 nM, respectively. Temperature was raised from 25° C. to 98° C. in 0.5° C. intervals over the course of 45 minutes, and fluorescence readings were taken at each interval. The fluorescence data were plotted and normalized, and the first derivative of the curve was calculated to provide the melting temperatures (Tm) using GraphPad Prism, version 5.0, as previously described (Yeh et al., 2006, Acta Crystallogr Section D, Biol Crystallogr 62:451-7).

While the invention has been described through embodiments, routine modifications to the disclosure here will be apparent to those skilled in the art. Such modifications are intended to be within the scope of this disclosure.

1. An immunogenic composition comprising:
(i) one or more of the following:
(a) a polypeptide AfeA having the sequence of SEQ ID NO:9
(b) a variant of the sequence of AfeA, said variant having a sequence that is at least 85% identical to the sequence of SEQ ID NO:9 and comprising at least one surface exposed epitope of AfeA,
(c) a fragment of AfeA, said fragment comprising at least 15 contiguous amino acids from the sequence of SEQ ID NO:9 and comprising at least one surface exposed epitope of AfeA; and/or
(d) a fragment of a variant of AfeA, said fragment of the variant comprising at least 15 contiguous amino acids from a sequence that has at least 85% identity to the sequence of SEQ ID NO:9, wherein the fragment of the variant comprises at least one surface exposed epitope of AfeA; and
(ii) an adjuvant.

2. The immunogenic composition of claim 1, wherein the fragment in (i)(c) or (i)(d) has from 15 to 30 amino acids.

3. The immunogenic composition of claim 1, wherein variant in (i)(b) or (i)(d) has at least a 90, 95, or 99% identity with SEQ ID NO:9.

4. The immunogenic composition of claim 1, further comprising one or more additional antigens from Moraxella catarrhalis.

5. The immunogenic composition of claim 4, wherein the one or more additional antigens from Moraxella catarrhalis are selected from the group consisting of CysP, SBP2, OppA and OMP CD.

6. The immunogenic composition of claim 1, further comprising one or more antigens from bacteria selected from the group consisting of Haemophilus influenza, Streptococcus pneumoniae and Moraxella catarrhalis.

7. A method of immunizing an individual against Moraxella catarrhalis infection comprising administering to the individual a composition of claim 1.

8. The method of claim 7, wherein the individual is a child of age 2 years of less.

9. The method of claim 7, wherein the individual is a child of age above 2 years to 18 years of age.

10. The method of claim 7, wherein the individual is an adult.

11. The method of claim 7, further comprising administering one or more booster doses of the composition.

12. A method of reducing the risk of otitis media in a child comprising administering to the child one or more times a composition of claim 1.

13. A method of reducing the risk of exacerbations of chronic obstructive pulmonary disease in an adult comprising administering to the adult one or more times a composition of claim 1.

14. A kit for immunizing an individual against Moraxella catarrhalis infections comprising one or more doses of the composition of claim 1, packaged together with a drug delivery device and optionally, instructions for use.
